(19) United States
(12) Patent Application Publication
(10) Pub. No.: US 2006/0041946 A1
(43) Pub. Date: Feb. 23, 2006

(54) Nuclear transfer nuclei from histone hypomethylated donor cells

(76) Inventor: Adama G. Fisher, London (GB)

Correspondence Address: DARBY & DARBY P.C., P. O. BOX 5257, NEW YORK, NY, 10150-5257, US

(21) Appl. No.: 10/517,216

(22) PCT Filed: May 23, 2003
(86) PCT No.: PCT/GB03/02266

§ 371 Date: Mar. 22, 2005

(30) Foreign Application Priority Data
May 23, 2002 (GB) 0211904.8

(51) Int. Cl. A01K 67/00; C07K 5/06

(52) U.S. Cl. 800/8; 435/325

(57) ABSTRACT

The present invention provides a method of producing an animal embryo, the method comprising transferring from a nuclear donor cell which has been selected on the basis that it is histone hypomethylated at least a portion of the nuclear contents including at least the minimum chromosomal material able to support development into a suitable recipient cell.

The present invention relates to cloning procedures in which cell nuclei are transplanted into recipient cells. The nuclei are reprogrammed to direct the development of cloned embyros, which can then be transferred into recipient females to produce foetuses and offspring or used to produce embryonic cell lines.

All publications, patents and patent applications cited herein are incorporated in full by reference.

### BACKGROUND

A fundamental question in cell and developmental biology concerns how nuclei progressively acquire differentiated functions. Although the nucleus of a fertilised egg is totipotent in that all of the differentiated cell types found in the adult organism can be derived from it, this is not the case for the vast majority of somatic nuclei in the adult animal. This limitation of the genomic potential of nuclei is progressively acquired during embryonic and post-embryonic development. Although in most cells the DNA sequence content of nuclei remains unchanged as development proceeds, the repertoire of genes that are expressed in a given cell type becomes limited. It also becomes more difficult to reactivate genes that are silenced in that cell type. This limitation is now known to reflect the imposition of epigenetic regulatory mechanisms on genes, especially through the assembly of stable repressive nucleoprotein complexes in the differentiated cell nucleus. The molecular mechanisms necessary to stably repress genes are gradually established as embryogenesis and post-embryonic development proceed. Remarkably, the egg and oocyte can reverse this process of repression, disassembling repressive features of nuclear organisation and, in particular circumstances, recreating a state, of pluripotency and even totipotency.

Covalent modifications to histone proteins have been proposed as the basis for an epigenetic code capable of extending the information potential of primary DNA sequences [1]. This code could ‘mark’ the transcriptional status of genes and also provide a plausible self-templating mechanism to propagate chromatin status through DNA replication and mitosis. Transcriptionally active euchromatin and inactive heterochromatin have been characterized by generalized differences in histone modifications [2]. For example, there is a global under-acetylation (particularly of H4) in heterochromatin domains such as those exemplified by the inactive X chromosome in mammals [3]. In addition, more subtle site-specific changes are also consistent features of euchromatin versus heterochromatin. For example, acetylation at lysine 12 in H4 appears to be a hallmark of heterochromatin [4, 5] whereas acetylation of lysine 9 in H3 represents a euchromatic imprint in Tetrahymena and many other organisms (reviewed in [6]). Methylation of H3 at lysine residues 4 or lysine 9 is reciprocally associated with euchromatic or heterochromatic regions, respectively [7,1].

A discussion of methylation at lysine 9 of H3 in animals may be found in Cowell et al. (2002) Chromosomsa, 111:22-36. Contrary to the findings presented herein, Cowell et al. states that methylation at lysine 9 of H3 represents one of the most robust histone modifications and suggests that it is almost permanent in nature.

Although an increasing number of factors involved in transmitting gene expression patterns have been identified, we do not as yet know how, at a mechanistic level, transcriptional competence is conveyed to daughter cells. Polycomb (PcG) and Trithorax (TrxG) group proteins appear to be crucial for the clonal inheritance of the inactive and active state of target genes in diverse organisms [8, 9]. In addition, genes previously characterized as modifiers of position effect variegation (PEV) can also influence the transmission of epigenetic information [10-14]. These include some structural components of heterochromatin, such as HP1 (allelic to Su(var)2-5), as well as enzymes that modify histones, such as the Suv39h HMTases [14-17].

Interest in the basic molecular mechanisms involved in the imposition of epigenetic regulatory mechanisms on genes has been stimulated by the economic and medical implications of the cloning of animals by nuclear transfer from donor embryos and from adult cell nuclei. Unfortunately, the economic and medical exploitation of cloning technology has been hampered by the extremely low efficiency of cloning from adult cell nuclei with most clones dying during gestation.

Somatic nuclei can be reprogrammed by nuclear transfer into enucleated oocytes as originally described by Wakayama and colleagues in 1998. Although approximately 20-40% of renucleated oocytes develop to the blastocyst stage, in most case less than one percent result in live born animals suggesting that complete reprogramming is a rare event (reviewed in Yanagimachi, R. Mol Cell Endocrin. (2002) 187 p 241-248). Reprogramming can also be achieved by clear transfer into fertilised mouse eggs (Modlinski, J. A. 1978. Nature 273 p. 466-467). Although the latter technique results in tetraploidy of the resultant embryos, the technique itself is much simpler and more robust than traditional cloning (our observations) and allows the assessment of the differences in reprogram potential of multiple cell types.

Attempts to increase efficiency have included varying the source of donor nuclei. For instance, EP 930 009 describes the use of resting cells as nuclear donor cells whilst WO 99/53751 and Hoechedlinger and Jaenisch (2002) Nature, 415: 1035 to 1038 describes the use of lymphocytes as nuclear donors. However, Hoechedlinger and Jaenisch (2002) found that the use of lymphocytes as nuclear donor cells was relatively inefficient and concluded that the efficiency was about ten times lower than that from other donor cell populations. It was suggested that the low efficiency could be due to inefficient reprogramming of the lymphocyte genome or differences in the sensitivity of the lymphocyte nuclei to the nuclear transfer protocol.

In view of the foregoing, it will be appreciated that there is a need for an improved understanding of the mechanisms underlying epigenetic regulation and a need for new approaches towards improving the efficiency and success of nuclear transfer procedures.

### THE INVENTION

The present invention is based on the discovery that cells which have histone hypomethylation may advantageously be used as nuclear donor cells. By using cells which have histone hypomethylation the efficiency of nuclear transfer may be increased.

A first aspect of the invention provides a method of producing an animal embryo, the method comprising transferring from a nuclear donor cell which has been selected on the basis that it is histone hypomethylated at least a portion of the nuclear contents including at least the minimum chromosomal material able to support development into a suitable recipient cell.

By a “cell which has been selected on the basis that it is histone hypomethylated” we include:
(i) testing a cell to determine if it is histone hypomethylated and selecting the cell if it is found to be histone hypomethylated;
(ii) experimentally determining that a first cell is histone hypomethylated and selecting a second cell (the nuclear donor cell) which is similar or identical to the first cell to thereby select a histone hypomethylated cell to be used as a nuclear donor cell; and
(iii) selecting a histone hypomethylated cell by selecting a cell of a type which has been previously determined as being histone hypomethylated (e.g. a resting B lymphocyte, preferably a small resting B lymphocyte) or which has been previously determined as being likely to be histone hypomethylated.

Preferably technique (ii) or (iii) is used.

With respect to technique (ii), by the second cell being “similar” to the first cell we refer to the second cell being sufficiently similar to the first cell (i.e. having a sufficient number of characteristics in common) such that it is reasonable to infer that because the first cell exhibits histone hypomethylation the second cell also exhibits histone hypomethylation. Preferably, the first and second cells are from the same population of cells, such as a population of cells which has been enriched for histone hypomethylated cells.

By “experimentally determining that a first cell is histone hypomethylated and selecting a second cell (the nuclear donor cell) which is similar or identical to the first cell to thereby select a histone hypomethylated cell to be used as a nuclear donor cell” we include determining that a single first cell is histone hypomethylated and selecting a second cell which is similar or identical to the first cell.

By “experimentally determining that a first cell is histone hypomethylated and selecting a second cell (the nuclear donor cell) which is similar or identical to the first cell to thereby select a histone hypomethylated cell to be used as a nuclear donor cell” we also include determining that more than one first cell is histone hypomethylated and selecting a second cell which is similar or identical to the first cells. The first cells may be subjected to the same or different assay for histone hypomethylation. For instance, one first cell or one aliquot of first cells may be tested with one type of antibody and one second cell or one aliquot of second cells may be tested with a second type of antibody etc.

With respect to technique (iii), by a type of cell which has been previously determined as being likely to be histone hypomethylated we include a type of cell of which at least 70%, 80%, 90%, 95%, 99% or 99.5% of cells of that type are hypomethylated.

Advantageously, the nuclear donor cell has reduced expression or activity of one or more histone methyl transferases. Thus, in a preferred embodiment of the invention, the nuclear donor cell is selected on the basis that it has reduced expression or activity of one or more histone methyl transferases.

Preferably, the nuclear donor cell has reduced expression or activity of 1, 2, 3, 4, 5, 6, 7, 8, 9, 10 or 11 of the following enzymes capable of methylating lysine residues of histone H3 or histone H4: Suv39h1, Suv39h2, ESET, Ezh2, PR-set7, SET7/9, ASH1, ASH2, ALL1(trithorax), DOT1L and G9a.

By a “cell which has been selected on the basis that it has reduced expression or activity of a histone methyl transferase” we include:
(i) testing a cell to determine if it is has reduced expression or activity of a histone methyl transferase and selecting the cell if it is found to have reduced histone methyl transferase expression or activity;
(ii) experimentally determining that a first cell has reduced expression or activity of a histone methyl transferase and selecting a second cell (the nuclear donor cell) which is similar or identical to the first cell to thereby select a cell having reduced histone methyl transferase expression or activity to be used as a nuclear donor cell; and
(iii) selecting a cell having reduced expression or activity of a histone methyl transferase by selecting a cell of a type which has been previously determined as having reduced expression or activity of a histone methyl transferase or which has been previously determined as being likely to have reduced expression or activity of a histone methyl transferase.

Preferably technique (ii) or (iii) is used.

With respect to technique (ii), by the second cell being “similar” to the first cell type we refer to the second cell being sufficiently similar to the first cell (i.e. having a sufficient number of characteristics in common) such that it is reasonable to infer that because the first cell has reduced expression or activity of a histone methyl transferase the second cell also has reduced expression or activity of a histone methyl transferase.

By “experimentally determining that a first cell has reduced expression or activity of a histone methyl transferase and selecting a second cell (the nuclear donor cell) which is similar or identical to the first cell” we include determining that a single first cell has reduced expression or activity of a histone methyl transferase and selecting a second cell which is similar or identical to the first cell.

By “experimentally determining that a first cell has reduced expression or activity of a histone methyl transferase and selecting a second cell (the nuclear donor cell) which is similar or identical to the first cell” we also include determining that more than one first cell has reduced expression or activity of a histone methyl transferase and selecting a second cell which is similar or identical to the first cells.

With respect to technique (iii), by a type of cell which has been previously determined as being likely to have reduced expression or activity of a histone methyl transferase we include a type of cell of which at least 70%, 80%, 90%, 95%, 99% or 99.5% of cells of that type have reduced expression or activity of a histone methyl transferase.

Persons skilled in the art will readily be able to devise assays for determining the level of expression or activity of histone methyl transferases. Immunofluorescence-based approaches or protein-based technologies (ie. cells lysates and western blotting) may be used.

Expression of a histone methyl transferase may, for example, be assayed by using antibodies which detect the histone methyl transferase. Techniques for raising antibodies with desired specificities will be well known to those skilled in the art. Moreover, some antibodies with appropriate specificities are commercially available.

Activity of a histone methyl transferase may, for example, be assayed by using antibodies which detect methylated lysine residues. Techniques for raising antibodies with desired specificities will be well known to those skilled in the art. Moreover, some antibodies with appropriate specificities are commercially available. For example antibodies to 1× methyl H3-K9, methyl H3-K4 are available from Upstate Biotechnologies.

Histone methyl transferase activity can assessed by looking at the extent of incorporation of methyl groups into a specific histone substrate. This method has been published (for example ref 30, Kuzmichev et al., also the paper by Rea et al., 2000) and would be straightforward for someone skilled in the art.

Preferably, a “cell which has reduced expression or activity of a histone methyl transferase” has ≦50% (and more preferably ≦45%, 40%, 35%, 25%, 20%, 15%, 10%, 5%, 3%, 2% or 1%) of the average level of histone methyltransferase expression or activity of a population of activated or cycling cells of the same type (e.g. 24-hour activated, 48-hour activated or 72-hour activated cells).

In one embodiment of the invention, a cell may be treated to reduce the activity of a histone methyl transferase. In this way cells which are more suitable for use as a nuclear donor cell may be obtained. In one embodiment of the invention, the nuclear donor cell is a cell which has been genetically engineered to have a reduced activity of one or more histone methyl transferases. Preferably, a nuclear donor cell in which one allele of the HMTase gene(s) in question has/have been deleted/removed/inactivated (rather than both). Similarly, naturally occurring mutant cells having reduced activity of a histone methyl transferase may also be used.

Preferably, the nuclear donor cell is obtained by a method which comprises enriching a population of cells for suitable nuclear donor cells and selecting the nuclear donor cell from the enriched population.

Preferably, the enrichment process comprises separating histone hypomethylated cells from non-histone hypomethylated cells to thereby obtain a population enriched for histone hypomethylated cells.

Preferably, at least about 70%, 80%, 90%, 95%, 99%, or 99.5% of cells in the enriched population are histone hypomethylated. More preferably, about 100% of cells in the enriched population are histone hypomethylated.

In one embodiment of the invention, the enrichment process comprises separating histone hypomethylated cells (e.g. small resting B lymphocytes) from histone hypomethylated cells having higher levels of histone methylation (e.g. large resting B lymphocytes). In this way a population of cells having particularly low levels of histone hypomethylation (such as small resting B lymphocytes), and which are particularly suitable as nuclear donor cells, may be obtained. Preferably, at least about 70%, 80%, 90%, 95%, 99%, or 99.5% of the resulting cells are small resting B lymphocytes. More preferably, about 100% of cells in the enriched population are small resting B lymphocytes.

In a particularly preferred embodiment of the invention, a population of cells is enriched for resting B lymphocytes and a small resting B lymphocyte is selected from the population of cells enriched for resting B lymphocytes. A single small resting B lymphocyte may be selected or a population enriched for small resting B lymphocytes may be obtained from the population enriched for resting B lymphocytes. A small resting B lymphocyte may then be selected from the population of cells enriched for small resting B lymphocytes and be used as a nuclear donor cell. Preferably, at least about 70%, 80%, 90%, 95%, 99%, or 99.5% of the cells in the population enriched for small resting B lymphocytes are small resting B lymphocytes. More preferably, about 100% of cells in the population enriched for small resting B lymphocytes are small resting B lymphocytes.

Various criteria may be used to obtain a population enriched for suitable nuclear donor cells. For example, the cells may be separated on the basis of one or more physical criteria, such as size or density, or on the basis of resistance to enzymatic digestion (for example in the case of distinguishing hepatocytes and kupffer cells).

In a preferred embodiment, an enrichment step which differentiates between small resting B lymphocytes and large resting B lymphocytes is performed. Small and large B cells can be distinguished on the basis of nuclear diameter (measuring at the widest place, for example as measured by confocal microscopy). For example, in rodents such as mice, small resting B cells have a nuclear diameter of about 8.0 μm or less (preferably ≦7.0 μm, 6.5 μm 6.0 μm, 5.5 μm, 5.0 μm, 4.5 μm, 4.0 μm, 3.5 μm, 3.0 μm or 2.5 μm). Those skilled in the art will be able to determine empirically those nuclear diameters which may be used to characterise B cells of other species into small and large resting B cells. Similarly those skilled in the art will be able to determine empirically those nuclear diameters which may be used to characterise T cells of rodents and other species into small and large resting T cells.

Separation of resting B cells into small and large resting B cells may be done by density gradient separation, such as the method described by Ratcliffe and Julius [22]. The population of resting B lymphocytes which is separated into small and large resting B lymphocytes is preferably obtained by a method comprising CD43-depletion of cells, such as CD43-depletion of splenic cells.

In one preferred embodiment, the nuclear donor cell is a lymphocyte (e.g. B lymphocyte or T lymphocyte) and the nuclear donor cell is obtained by a method which comprises enriching for small resting B lymphocytes (and/or small resting T lymphocytes) and selecting the nuclear donor cell from the population of small resting B lymphocytes (and/or small resting T lymphocytes).

In a one embodiment of the invention, an enrichment step which differentiates between small resting T lymphocytes and large resting T lymphocytes is performed.

A population enriched for suitable nuclear donor cells may be obtained by enzymatic separation. In enzymatic separation, one or more enzymes are employed which digest unwanted cells. For example, kupffer cells may be obtained from a liver cell suspension comprising kupffer cells and hepatocytes by incubation with pronase (see the Examples section).

Preferably, one or more cells from the enriched population (e.g. enriched for resting B lymphocytes or small resting B lymphocytes) are tested for histone hypomethylation.

Preferably, one or more cells from the enriched population (e.g. enriched for resting B lymphocytes or small resting B lymphocytes) are tested for reduced histone methyl transferase expression.

In one embodiment of the invention, the cells present in a tissue section are tested for histone hypomethylation. In this manner, large numbers of cells can be tested to identify cells which are histone hypomethylated which may be useful as nuclear donor cells. This information may then be used to select a histone hypomethylated nuclear donor cell. Thus, as described in the examples section, antibody-labelling of liver sections allowed the identification of histone hypomethylated kupffer cells. An enrichment step for kupffer cells may then be performed (e.g. by enzymatic separation) and a nuclear donor cell selected from the resulting population of kupffer cells.

Histone hypomethylated cells may be readily distinguished from methylated cells due to the considerable and readily-observable differences in their respective levels of histone methylation. Indeed, the level of methylation in hypomethylated cells appears to be negligible or absent (or at least undetectable). Thus, for practical purposes methylation may generally be considered to be an all or nothing (or almost nothing) event. Accordingly, the skilled person will readily be able to appreciate whether a cell is hypomethylated or not.

Preferably, a cell is regarded as being histone hypomethylated if histone methylation is negligible or absent (absent being used herein to mean undetectable).

As demonstrated in the Example below, cells which are hypomethylated include G₀ (resting) lymphocytes and some liver cells (as indicated below these liver cells may be Kupffer cells). Also, the paternal genome, early after fertilisation has been identified as being hypomethylated (see Cowell et al. (2002)).

Cells which are not hypomethylated include activated lymphocytes, serum starved fibroblasts and some post-mitotic cells (e.g. cumulus cells and multinucleated muscle fibres).

Histone methylation may be assessed in various ways as outlined below.

The level of histone methylation can be assessed directly using antibodies that detect methylated lysine residues. The level of antibody binding is a direct reflection of the level of histone methylation in each cell. Techniques for raising antibodies with desired specificities will be well known to those skilled in the art. Moreover, some antibodies with appropriate specificities are commercially available. For example antibodies to 1× methyl H3-K9, methyl H3-K4 are commercially available from Upstate Biotechnologies. See also the Materials and Methods section below.

To assess histone methylation, immunofluorescence-based approaches or protein-based technologies (ie. cells lysates and western blotting) may be used (see the Examples section below).

Histone methylation may be assessed with regard to one or more histone types. Preferably, the level of histone methylation is assessed with regard to H3 and/or H4, preferably with regard to H3.

The assessment of histone methylation may involve assessing whether one or more histone residues are methylated. Obviously, for the assay to be meaningful only methylation at histone residues which are known to undergo histone methylation is assessed.

With regard to techniques (ii) and (iii), in one embodiment it is preferred that the level of histone methylation of said first cell or of said cell type is assessed on the basis of methylation at one or more residues of H3.

Histone residues which may be methylated include lysine and arginine residues. In one embodiment, methylation at one or more lysine residues is assessed. In another embodiment methylation at one or more arginine residues is assessed. Preferably, methylation at one or more lysine residues and at one or more arginine residue is assessed.

Lysine residues of H3 which may be methylated in mammals include residues 4, 9, 27 and 36 (Rice and Allia (2001) Current Opinion in Cell Biology, 13:263-273; and Richards and Elgin (2002) Cell 108, 489-500). Preferably, methylation at one, two, three or four of these lysine residues is assessed. Preferably, methylation of H3^{K4} or H3^{K9} or H3^{K27} is assessed.

Preferably, methylation of both H3^{K4} and H3^{K9} is assessed. Preferably, methylation of H3^{K4}, H3^{K9} and H3^{K27} is assessed.

Preferably, methylation at ≧two, three, four, five or six histone residues is assayed.

The assessment of histone methylation may involve assessing the extent of methylation (ie. mono-, di- or tri-methylation) at one or more residue(s).

It will be appreciated that an assessment of histone methylation may involve assaying different histones for methylation and/or different histone residues for methylation and/or the extent of methylation at different residues.

Preferably, a cell which is regarded as being histone hypomethylated has negligible or absent (i.e. undetectable) methylation at ≧one, two, three, four, five or six histone residues.

In one embodiment, a cell is regarded as being histone hypomethylated if it has 10% or less (and more preferably ≦8%, 5% or 2%) of the level of histone methylation of one or more (and preferably any) of the cell types listed above as examples of cells which are not hypomethylated. Preferably, a cell is regarded as being histone hypomethylated if it has 10% or less (and more preferably ≦8%, 5% or 2%) of the level of histone methylation of an activated or cycling lymphocyte.

Methods of quantifying histone methylation will be known to those skilled in the art or can be readily devised by those skilled in the art. For example, a semi-quantitative western blotting approach may be used.

In one embodiment of the invention, a cell is regarded as being histone hypomethylated if it has ≦50% (and more preferably ≦45%, 40%, 35%, 25%, 20%, 15%, 10%, 5%, 3%, 2% or 1%) of the average level of histone methylation of a population of activated or cycling cells of the same type (e.g. 24-hour activated, 48-hour activated or 72-hour activated cells).

The level of histone hypomethylation may be assessed as described herein in relation to FIG. 7a. Briefly, the level of histone methylation may be assessed by comparing the cellular intensity of a mono- di- or tri- methylated H3-K9 labelled test cell with the average level of cellular intensity of a mono- di- or tri- methylated H3-K9 labelled population of activated (or cycling) cells. Following labeling of the cells with antibodies specific to mono- di- or tri- methylated H3-K9, confocal images of the cells are taken at identical settings for each antibody studied. The total labeling of the nucleus of each cell can then be quantitated calculating either the average pixel intensity of each nucleus (pixel average) or the total intensity of each nucleus (integrated). The technique can be used to obtain a reading for an individual “test” cell and this reading may then be compared with the average reading (preferably the mean of ≧about 25, 75, 150 cells) obtained for a number of “benchmark” cells which may be activated or cycling cells.

The term “embryo” as used herein includes all concepts of an animal embryo such as an oocyte, egg, zygote or an early embryo. More specifically, the term “embryo” used herein includes morulas (8-16 cells), morulas (16-32 cells) and blastocysts (64 cells and above).

The term “nuclear donor cell” as used herein includes a cell from which at least a portion of the nuclear contents including at least the minimum chromosomal material able to support development is transferred into a suitable recipient cell. Similar expressions e.g. “nuclear transfer” should be interpreted in a likewise manner.

Preferably, the nuclear donor cell employed in the present invention is a mammalian cell. Preferably, the recipient cell is a mammalian cell. Preferably, the nuclear donor cell and the recipient cell are both mammalian cells; preferably they are both ungulate, rat or murine cells.

In an alternative embodiment the nuclear donor cell and/or recipient cell is not a mammalian cell. The nuclear donor cell and/or recipient cell may, for example, be a Xenopus cell.

Preferably, donor cells and recipient cells from the same species are used. Preferably, the donor cell and recipient cell are both human cells or mouse cells.

Cells derived from populations grown in vivo or in vitro and containing 2n chromosomes (e.g. those in G0 or G1) or greater than 2n chromosomes (e.g., those in G2, which are normally 4n) may act as nuclear donor cells.

An example of an in vivo source of the 2n donor nucleus is a cumulus cell. One embodiment of the invention contemplates using donor nuclei taken from either in vivo or in vitro (i.e., cultured) sources of 2n adult somatic cells including, without limitation, epithelial cells, neural cells, epidermal cells, keratinocytes, hematopoietic cells, melanocytes, chondrocytes, B or T lymphocytes, macrophages, monocytes, nucleated erythrocytes, fibroblasts, Sertoli cells, cardiac muscle cells, skeletal muscle cells, smooth muscle cells, and other cells from organs including, without limitation, skin, lung, pancreas, liver, kidney, urinary bladder, stomach, intestine, bone, and the like, and their progenitor cells where appropriate.

In one embodiment, the donor cell is a resting cell (G₀), preferably a resting B lymphocyte or resting T lymphocyte, preferably a small resting T lymphocyte. Preferably, the nuclear donor cell is a small resting B lymphocyte obtained or obtainable by density gradient separation, such as described above or as in the Examples section.

In another embodiment of the invention, the donor adult somatic cell is “2-4C”; that is, it contains one to two times the diploid genomic content, as a result of replication during S phase of the cell cycle. This donor cell may be obtained from an in vivo or an in vitro source of actively dividing cells including, but not limited to, epithelial cells, hematopoietic cells, epidermal cells, keratinocytes, fibroblasts, and the like, and their progenitor cells where appropriate.

In one embodiment of the invention it is preferred that the donor cell is not selected from the group consisting of: a resting lymphocyte, a resting B lymphocyte, a liver cell, or a Kupffer cell.

Optionally the donor nucleus may be genetically modified. The donor nucleus may contain one or more transgenes and the genetic modification may take place prior to nuclear transfer and embryo reconstitution. Such a genetically modified donor nucleus may be used in the creation of a transgenic animal.

It should be noted that the term “transgenic”, in relation to animals, should not be taken to be limited to referring to animals containing in their germ line one or more genes from another species, although many transgenic animals will contain such a gene or genes. Rather, the term refers more broadly to any animal whose germ line has been the subject of technical intervention by recombinant DNA technology. So, for example, an animal in whose germ line an endogenous gene has been deleted, duplicated, activated or modified is a transgenic animal for the purposes of this invention as much as an animal to whose germ line an exogenous DNA sequence has been added.

Preferably, the recipient cell is a one cell zygote, enucleated oocyte, embryonic stem (ES) cell or any other type of cell which may facilitate in the reprogramming of the donor nucleus. As will be appreciated from below, the recipient cell may be the “ultimate” recipient cell in which case the resulting embryo may directly give rise to a foetus or animal (offspring). Alternatively, in the case of serial nuclear transfer (discussed below), the recipient cell may not be the “ultimate” recipient cell and it may act as a nuclear donor cell.

Preferably, the enucleated oocyte is a mammalian enucleated oocyte. Enucleation may be achieved physically, by actual removal of the nucleus, pro-nuclei or metaphase plate (depending on the recipient cell), or functionally, such as by the application of ultraviolet radiation or another enucleating influence.

Oocytes that may be used in the method of the invention include both immature (e.g., GV stage) and mature (i.e., Met II stage) oocytes. Mature oocytes may be obtained, for example, by inducing an animal to super-ovulate by injections of gonadotrophic or other hormones (for example, sequential administration of equine and human chorionic gonadotrophins) and surgical harvesting of ova shortly after ovulation (e.g., 80-84 hours after the onset of estrous in the domestic cat, 72-96 hours after the onset of estrous in the cow and 13-15 hours after the onset of estrous in the mouse). Where it is only possible to obtain immature oocytes, they are cultured in a maturation-promoting medium until they have progressed to Met II; this is known as in vitro maturation (“IVM”). Methods for IVM of immature bovine oocytes are described in WO 98/07841, and for immature mouse oocytes in Eppig & Telfer (Methods in Enzymology 225, 77-84, Academic Press, 1993).

Preferably, the recipient cell to which the donor cell nucleus is transferred is an enucleated metaphase II oocyte, an enucleated unactivated oocyte or an enucleated preactivated oocyte. At least where the recipient is an enucleated metaphase II oocyte, activation may take place at the time of transfer. Alternatively, at least where the recipient is an enucleated unactivated metaphase II oocyte, activation may take place subsequently.

Once suitable donor and recipient cells have been selected, it is necessary for the nuclear material of the former to be transferred to the latter. The nuclear donor cell can be transferred intact into a suitable recipient cell, optionally with a broken cell membrane. Alternatively, the nuclear contents of the donor cell (or a portion of the nuclear contents including at least the minimum chromosomal material able to support development) can be directly inserted into the cytoplasm of an enucleated oocyte.

Conveniently, nuclear transfer is effected by fusion. Three established methods which have been used to induce fusion are: (i) exposure of cells to fusion-promoting chemicals, such as polyethylene glycol; (ii) the use of inactivated virus, such as Sendai virus; and (iii) the use of electrical stimulation.

Alternatively, nuclear transfer is effected by microinjection.

Before or (preferably) after nuclear transfer (or, in some instances at least, concomitantly with it), it is generally necessary to stimulate the recipient cell into development by parthenogenetic activation, at least if the cell is an oocyte. In one embodiment, the activation step takes place from zero to about six hours after nuclear transfer in order to allow the nucleus to be in contact with the cytoplasm of the oocyte for a period of time prior to activation of the oocyte. Activation may be achieved by various means which will be well known to those skilled in the art.

There are several options for which the embryos made by the present invention may be used for.

In one embodiment, the embryo may be used in serial nuclear transfer. Thus, a second aspect of the invention provides a method of producing an animal embryo, the method comprising transferring from a nuclear donor cell at least a portion of the nuclear contents including at least the minimum chromosomal material able to support development into a suitable recipient cell wherein the nuclear donor cell is obtained from an embryo obtained by the method of the first aspect of the invention.

Preferably, the nuclear donor cell obtained from an embryo obtained in accordance with the first aspect of the invention has been selected on the basis that it is histone hypomethylated.

It will be appreciated that the embryo obtained by the method of the second aspect of the invention may be used for further rounds of serial nuclear transfer.

Preferably, an embryo obtained by the first or second aspects of the invention is allowed to develop into a foetus or animal (i.e. live offspring). Thus, a third aspect of the present invention provides a method of producing a foetus the method comprising allowing an embryo obtained by the first or second aspect of the invention to develop into a foetus.

The step of allowing the embryo to develop may include the substep of transferring the embryo to a female mammalian surrogate recipient, wherein the embryo develops into a viable foetus. The embryo may be transferred at any stage, including from the two-cell to morula/blastocyst stage, as known to those skilled in the art.

A fourth aspect of the invention provides a method of producing a non-human animal the method comprising allowing an embryo obtained by the first or second aspects of the invention or a foetus obtained by the third aspect of the invention to develop into said non-human animal.

Those skilled in the art will appreciate that the cloned embryos of the present invention may be combined with fertilized embryos to produce chimeric embryos, foetuses and/or offspring. Such chimeric embryos, foetuses and/or offspring are also included within the scope of the present invention.

In another aspect of the invention an embryo of the present invention is used in the preparation of an embryonic stem cell line. Thus, a fifth aspect of the present invention provides a method of producing an embryonic stem cell line, the method comprising transferring an embryo obtained by the method of the first or second aspect of the invention to a culture system.

A sixth aspect of the invention provides a method of producing an embryonic stem cell line, the method comprising isolating the inner cell mass of an embryo obtained by the method of the first or second aspect of the invention and transferring the inner cell mass to a culture system.

An embryonic cell line could find beneficial application in its use to generate embryonic stem cells from a patient as a source of compatible undifferentiated cells to be used in transplantation for the therapy of degenerative diseases.

In an seventh aspect of the invention, a cell could be treated to artificially reduce the level of histone methylation so as to render the cell histone hypomethylated. The cell could be employed as a nuclear donor cell in the above described methods of the present invention. The treatment may be chemical or enzymatic and may, for example, involve treatment with a histone demethylase or with a histone methyltransferase (HMT) inhibitor¹.

An eighth aspect of the invention relates to the embryos, foetuses, non-human animals, and embryonic cells obtained by the methods described above.

A ninth aspect of the invention relates to the use of histone hypomethylation status as an indicator of the suitability of a cell to act as a nuclear donor cell. Histone hypomethylation status may be assessed as described above.

A tenth aspect of the invention provides a method of selecting a cell to be used as a nuclear donor cell the method comprising selecting said cell on the basis that it is histone hypomethylated.

An eleventh aspect of the invention relates to the use of a resting B or T lymphocyte or Kupfer cell as a nuclear donor cell. Preferably, the resting B or T lymphocyte is a small resting B or T lymphocyte.

Preferably, the small resting B lymphocyte is obtained by a method comprising selecting a small B lymphocyte from a population of cells comprising resting B lymphocytes.

Preferably, the small resting B lymphocyte is obtained by a method comprising selecting a small B lymphocyte from a population of cells enriched for resting B lymphocytes. Preferably, one or more of the cells in the population of cells enriched for resting B lymphocytes are tested for histone hypomethylation.

Preferably, one or more of the cells in the population of cells enriched for resting B lymphocytes are tested for reduced expression or activity of a histone methyl transferase. Preferably, at least about 70%, 80%, 90%, 95%, 99%, or 99.5% of cells in the population enriched for resting B lymphocytes are histone hypomethylated. Preferably, at least about 100% of cells in the population enriched for resting B lymphocytes are resting B lymphocytes.

In one embodiment, the population of cells enriched for resting B lymphocytes is obtained by CD43-depletion of splenic cells.

Preferably, the small B lymphocyte is obtained from the population of cells enriched for resting B lymphocytes by visually detecting a small resting B lymphocyte cell present in the enriched population and selecting the cell.

Alternatively, the small B lymphocyte is obtained from the population of cells enriched for resting B lymphocytes by obtaining a population of cells enriched for small resting B lymphocytes from the population of cells enriched for resting B lymphocytes and selecting a small B lymphocyte from the population of cells enriched for small resting B lymphocytes.

Preferably, one or more of the cells in the population of cells enriched for small resting B lymphocytes are tested for histone hypomethylation.

Preferably, one or more of the cells in the population of cells enriched for small resting B lymphocytes are tested for reduced expression or activity of a histone methyl transferase.

Preferably, at least about 70%, 80%, 90%, 95%, 99%, or 99.5% of cells in the population enriched for small resting B lymphocytes are small resting B lymphocytes. Preferably, at least about 100% of cells in the population enriched for resting B lymphocytes are small resting B lymphocytes.

Preferably, the population of cells enriched for small resting B lymphocytes is obtained from the population of cells enriched for resting B lymphocytes by density gradient separation.

In principle, the invention is applicable to all animals, including birds, such as domestic fowl, amphibian species and fish species. In practice, however, it will generally be to placental mammals that the greatest commercially useful applicability is presently envisaged. It is with ungulates, particularly economically important ungulates such as cattle, sheep, goats, water buffalo, camels and pigs that the invention is likely to be most useful, both as a means for cloning animals and as a means for generating transgenic or genetically modified animals. It should also be noted that the invention is also likely to be applicable to other economically important animal species such as, for example, horses, llamas or rodents e.g. rats, mice, rabbits and humans. However, due to ethical considerations, it may be desirable for certain aspects of the invention not to be applied to humans.

The present invention will now be described by reference to the accompanying Examples which are provided for the purposes of illustration and are not to be construed as being limiting on the present invention.

### BRIEF DESCRIPTION OF THE FIGURES

FIG. 1. HP1β (M31) and Ikaros proteins are up-regulated and redistributed to constitutive heterochromatin in B lymphocytes following mitotic stimulation. In (a) the kinetics of CD69 expression and BrdU incorporation by purified G₀ mouse B lymphocytes following mitotic stimulation with anti-IgM and CD40 antibodies is shown. Cells were sampled 0, 24, 48 and 72 hours (hrs) post-stimulation and the results show representative histograms of CD45RA (B220), CD69 and anti-BrdU labeling against cell number. In (b) the upper panels show representative confocal images of the nucleus of B lymphocytes simultaneously labeled with anti-Ikaros and anti-HP1β (M31) at 0, 24 and 72 hours post-stimulation. The nuclear periphery of each cells is outlined by lamin B labeling. In the lower panels, confocal images of lymphocytes co-stained with CREST anti-sera and DAPI are shown for comparison.

FIG. 2. Ikaros, HP1β Ezh2 and Bmi1 proteins are selectively up-regulated in the nucleus of B lymphocytes following mitotic stimulation. Panel (a) shows western blots in which the abundance of specific proteins within cytoplasmic (CE), soluble and insoluble nuclear extracts (NE-s and NE-i, respectively) are compared at different times after B lymphocyte activation. In panel (b) representative confocal images of the distribution of Ezh2, Eed, Bmi1 and ESET proteins relative to PI-labeled in the nuclei of quiescent (0 hrs) and cycling (72 hrs) B lymphocytes are shown.

FIG. 3. Selective increase in histone methylation in B lymphocytes following mitotic stimulation. Panel (a) shows the distribution of methylated H3-K9, H3-K4, H3-K27, acetylated H3-K9, H3-K14 or H4 in quiescent (0 hrs) and cycling (72 hrs) B cells measured by immunofluorescence, relative to DAPI labeling. Panel (b) shows the relative abundance of these modified histones estimated by western blotting of protein lysates harvested 0, 24 and 72 hrs after lymphocyte stimulation.

FIG. 4. H3-K9 methylation in resting and cycling B lymphocytes. In (a) the distribution of mono- (Me) di- ((Me)₂) and tri- ((Me)₃) methylated histone H3 in quiescent (0 hrs) and cycling (72 hrs) B cells is shown relative to DAPI labeling. Panel (b) shows the relative distribution of tri-methyl H3-K9, HP1β and DAPI labeling in resting (0 hrs) and cycling (72 hrs) B cells.

FIG. 5. Hypomethylation of histone H3 in liver Kupffer cells. In (a) mouse adult liver sections labeled with 4xmethyl H3-K9 and DAPI, a population of cells lacking methylated H3-K9 were seen (arrowed). Panel (b) shows labeling of isolated liver cell suspensions with biotinylated CD45 antibody revealed with avidin FITC either alone (left) or co-staining with antibody to methylated H3-K9 (4xmethyl H3-K9) or methylated H3-K4. Panel (c) shows methylated H3-K9 labeling (4xmethyl H3-K9) of freshly isolated Kupffer cells (0 hours) and following mitotic stimulation (24 hours in GM-CSF and IL-3)(upper panels), where Kupffer cells were identified by co-labeling with anti-CD45 (lower panels).

FIG. 6: Histone H3-K9 methylation is absent (or low) in quiescent mouse B lymphocytes and dynamically up-regulated upon mitotic stimulation. Methylated H3-K9 in the nucleus of G₀ (0 hrs) and cycling (72 hrs) B lymphocytes is shown (4xmethyl H3-K9 labeling) relative to DAPI-intense regions in the nucleus of cells isolated from normal male wild type) and Suv39h-deficient (left and right-hand columns, respectively). The distribution of Ikaros proteins in the nucleus of cycling B lymphocytes was not affected by the absence of Suv39h HMTases, as shown in the lower panels.

FIG. 7: Cellular intensities of mono- di- and tri- methylated H3-K9 labeling of resting and activated B cells. Following labeling of resting (day 0), 24 hour activated (day 1) and 72 hour activated B cells (day 3) with antibodies specific to mono- di- and tri-methylated H3-K9 (panel a). Confocal images of between 300-400 cells were taken at identical settings for each antibody studied. The total labeling of the nucleus of each cell was then quantitated calculating either the average pixel intensity of each nucleus (pixel average) or the total intensity of each nucleus (integrated). Individual events were then grouped into discrete quanta of intensities and expressed in histograms of increasing quanta of intensities (x axis) against cell number (y axis). The nuclear density of labeling of tri-methylated H3-K9 appeared low as labeling was concentrated to discrete foci. To allow for this, the proportion of cells that showed focussed H3-K9 labeling at each time point was assessed and is shown beneath the representative images shown in (b).

FIG. 8: Reduced H3-K9 methylation in resting versus cycling T lymphocytes Lymphocytes were isolated from the lymph nodes of mice (0 hrs) and T cells were stimulated by incubation on culture dishes coated with anti-TCR β chain antibody (H57, Pharmingen), in media supplemented with anti-CD28 (Pharmingen) and IL-2 for 3 days (72 hrs). T cells in these populations were identified by surface staining with APC-coupled anti-TCR β chain (H57, Pharmingen), and the samples were then fixed with 2% paraformaldehyde and IF analysis performed using antibody specific for M31/HP1β, and DAPI. The images show four cells, three of which are positive for surface T cell receptor (TCR) and can therefore be clearly identified as T cells. In resting T cells (left panels) an absence of M31 at pericentric heterochromatin (visualised by intense DAPI label) is apparent. However, in activated T cells M31 is redistributed to pericentric heterochromatin and co-localises with DAPI-bright areas.

FIG. 9: Reversal of transgene silencing is more efficient using donor nuclei from G₀ cells than activated B cells. Nuclei from resting (0-24 hours) or active (48-72 hours) B cells carrying the silent EGFP transgene were transferred into fertilised embryos 18-21 hours post injection with human chorionic gonadotrophin (BL6/D2×BL6/D2). Embryos surviving the transfer procedure were cultured overnight in M16 media (Specialty Media). On day one after transfer, 2-cell embryos were transferred into glucose-supplemented CZB media. The majority of operated embryos reached morulae or early blastocyst stage but were somewhat developmentally retarded as compared to control embryos. On day four after transfer, GFP expression was assessed. Consistently, twice as many embryos showed strong GFP expression after transfer with resting B cell nuclei as compared with activated B cells. Data from various experiments are shown.

FIG. 10: Heterogeneity of methylation patterns in resting B cells. Increased amounts of methylation were observed in large resting B cells as compared with small resting B cells.

FIG. 11: Peptide-blot analysis determining the specificity of antibodies used in this study. Peptides representing histone H3N-termini either mono-, di-, or trimethylated at the indicated position were transferred onto nitrocellulose in quantities of either 50, 10 or 2 pmoles. These blots were probed with the respective antibodies at the indicated concentrations. Binding efficiency was finally determined by a staining reaction of a secondary peroxidase coupled antibody (Jackson Immuno Research Laboratories).

Four antibodies raised against a 2×-branched peptide and one raised against a 4×-branched peptide were generated within the group of Thomas Jenuwein (left panel), whereas the two antibodies raised against a linear peptide are commercially available from Upstate Biotechnologies (UBI) (right panel). Both the UBI “linear” α-dimethyl H3-K9 antibody and the “4×-branched” α-dimethyl H3-K9 antibody have a major reactivity against methylated lysine 9 of the histone H3 tail but also have reactivity against methylated lysines 4 and 27 of histone H3. The UBI “linear” α-dimethyl H3-K4 antibody and “2×-branched” α-mono, di- and tri-methyl H3-K9 antibodies and the α-tri-methyl H3-K27 antibody display specific binding activity.

Abbreviations

BrdU Bromodeoxyuridine
CE Cytoplasmic extract
ChIP Chromatin Immunoprecipitation
DAPI 4′,6-Diamidino-2-phenylindole
HMTases Histone methyl transferases
HP1 Heterochromatin protein 1
IgM Immunoglobulin M
IL-3 Interleukin 3
IL-4 Interleukin 4
IF Immunofluorescence
Me Mono-methyl
(Me)₂ Di-methyl
(Me)₃ Tri-methyl
NE-i Nuclear extract insoluble
NE-s Nuclear extract soluble
PcG Polycomb group
PEV Position Effect Variegation
PRC1 Polycomb repressive complex 1
TCR T cell receptor
TrxG Trithorax group

### EXAMPLES

### SUMMARY

### BACKGROUND

Covalent modification of histones has been proposed as a possible mechanism of epigenetic inheritance based on observations that different patterns of histone methylation and acetylation are predictably associated with distinct chromatin and transcriptional states. To investigate their role in transcriptional memory, the extent of histone H3 and H4 modification in quiescent (G₀) and actively cycling mouse B lymphocytes was examined.

Results

We observed a generalised reduction in histone H3 methylation at lysine residues 4 (H3-K4), 9 (H3-K9) and 27 (H3-K27) in purified G₀ splenic B cells and the absence of heterochromatin-associated proteins HP1β and Ikaros at centromeric heterochromatin. Mitogenic stimulation resulted in a rapid increase in methylation at all three histone H3 residues prior to the onset of DNA replication, coincident with an up-regulation and global redistribution of Polycomb group proteins Bmi1, HP1 and of the Ezh2 and ESET MTases. Histone hypomethylation was also evident among non-cycling populations of Kupffer cells (but not hepatocytes) in adult liver and was reinstated following mitotic stimulation.

Conclusions

These results suggest that global methylation of histone H3 is more dynamic than had been previously appreciated and that histone hypomethylation is a feature of specific G₀ populations in vivo.

Introduction to Experimental Work

Here we investigate the contribution of histone modifications to epigenetic memory by comparing the extent of histone acetylation and methylation between purified resting (G₀) and cycling B lymphocytes. The rationale for this comparison lies with the capacity of quiescent lymphocytes to survive for extensive periods in vivo, but only re-enter the cell cycle upon antigenic stimulation. This implies that epigenetic information that defines both the lineage and developmental stage of differentiated B cells is actively retained in long-term quiescent cells. Consistent with this assumption, it is noteworthy that lymphocyte proliferation is severely impaired in mice lacking several individual PcG proteins [18-20]. We have previously shown that quiescent B lymphocytes lack some features found in cycling cells, most noticeably a lack of spatial association of transcriptionally inactive genes and Ikaros proteins at pericentric heterochromatin [21]. Here we directly compared histone modifications between cycling and non-cycling lymphocytes in order to assess the role of this putative ‘code’ in conveying cellular memory. Surprisingly, levels of H3-K4, H3-K9, H3-K27 methylation and Ezh2 and ESET HMTases were reduced or not detectable in quiescent primary B cells. These data show that chromatin composition differs significantly between resting and cycling cells and suggest that histone methylation is not necessarily a stable epigenetic imprint.

Materials and Methods

Purification and Activation of Resting B Lymphocytes from Spleen

Resting B Cell Purification from Spleen

Spleens of young (6-10 week old) mice were dissected and minced to yield single cell suspensions. Erythrocytes in this population were removed by treatment with Geyes solution (to lyse erythrocytes). Geyes solution was prepared by mixing 20 parts stock solution A (650 mM NH₄Cl, 25 mM KCl, 4 mM Na₂HPO₄.12H₂O, 1 mM KH₂PO₄, 28 mM Glucose) to 5 parts stock solution B (20 mM MgCl₂.6H₂O, 6 mM MgSO₄.7H₂O, 30 mM CaCl₂) to 5 parts stock solution C (267 mM NaHCO₃) to 70 parts sterile distilled water. To lyse erythrocytes, single cell suspensions were mixed with Geyes solution in a 1:4 ratio, and held on ice for 2 minutes before washing in media.

To remove CD43-positive cells from the cell suspension, cells were washed with cold (4° C.) buffer (0.5% BSA in PBS A) cells were incubated with anti-CD43 (Ly-48)-coupled micro beads (Miltenyi Biotech) in buffer according to manufacturers' instructions. Labelled (CD43-posistive) cells were washed in buffer and passed through a magnetised depletion column (Miltenyi Biotech). The column retains all paramagentically labelled cells but allows unlabelled (CD43-negative) cells to pass through.

Where stated the CD43 negative B cells were enriched by density gradient separation [21]. Where density gradient separation was used, CD43-negative cells were separated on a discontinuous Percoll gradient prepared and utilised as described previously (Ratcliffe and Julius, 1983). Briefly CD43-negative cells were applied to a discontinuous Percoll gradient (prepared with density steps 1.060, 1.079, 1.085, 1.092 and 1.109 g/ml) and small resting B cells were recovered at the 1.079-1.085 g/ml density interface following centrifugation (30 minutes at 1500 g). In this way small resting B cells could be prepared. B cell activation was induced by culturing cells in IMDM media containing 10% fetal bovine serum (Sigma) and antibiotics and 20 μg/ml purified anti-CD40 (monoclonal antibody FGK45), 101 g/ml purified anti IgM (monoclonal antibody H3074) and 2% IL-4 containing supernatant (from a T-helper cell line). Fluorescein-labeled antibodies to B220 and CD69 (BD Pharmingen) were used for FACs analysis to verify the phenotype and activation status of cells.

BrdU incorporation studies were performed using ex vivo resting mature B cells. Cells were cultured in media containing 50 μM BrdU with either IL-4 for 24 hours (for un-stimulated cells) or following activation using anti-IgM, anti-CD40 and IL-4 as described above. BrdU incorporation was revealed as previously described [54]; cells were fixed in ice cold 70% EtOH overnight at 4° C., washed in ice cold PBS, denatured in 3M HCl with 0.5% Tween for 20 min, followed by incubation in 0.01M sodium tetraborate solution for 3 min. After washing (2× in ice cold PBS) the cells were incubated in FITC-conjugated anti-BrdU monoclonal antibody (BD Pharmingen) before being washed and analysed by flow cytometry using a FACScan (Becton Dickinson).

Preparation of Liver Sections and Cell Suspensions

Liver sections were prepared and labeled as outlined elsewhere. Liver cells were prepared using a two-step Procedure previously described (Seglen, 1972, Exptl Cell Res 74 p 450; Seglen, 1998 Cell Biology: a laboratory handbook, Volume 1, p 119) with minor modifications. Whole liver was isolated from a recently sacrificed mouse, washed and a 25 g needle was inserted into the vena cava. Blood was rinsed from the liver by continuous perfusion with a large volume of pre-perfusion buffer (0.5 mM EGTA, 0.142M NaCl, 0.007M KCl, 0.01M HEPES, pH 7.4) until the tissue assumed a light tanned appearance. Pre-warmed (37° C.) collagenase buffer (0.5 mg/ml Collagenase (Sigma), 0.067M NaCl, 0.007M KCl, 0.005M CaCl₂.2H2O, 0.1M HEPES, pH 7.6) was then introduced through the vena cava continuously for 5-10 minutes until the structure of the tissue began to disintegrate. The tissue remnants were transferred into fresh media and single cells were liberated by gentle agitation. The resulting cell suspension (which contains hepatocytes and Kupffer cells) was passed through a 25 g needle washed twice in chilled media.

Isolation of non-parenchymal (kupffer cells) from liver cell suspensions can be achieved either by pronase digestion or by density gradient separation.

Pronase Digestion of Liver Cell Suspensions

Liver cell suspensions were incubated with 0.1% pronase (Sigma) for 1 hour at 37° C. (as per Seglen P O: Preparation of isolated rat liver cells. In: Methods in Cell Biology. pp. 29-83; 1976: 29-83.p 74) which digests/kills hepatocytes (cellular debris was removed by repeated washing and centrifugation). As this method can induce the activation of non-parenchymal cells an alternative (non-enzymatic) method is preferred.

Density Gradient Enrichment of Kupffer Cells from Liver Cell Suspensions

As non-parenchymal cells are less dense than most parenchymal cells, these can be separated on a discontinuous metrizamide (or percoll) gradient (an example is given in Seglen P O: Preparation of isolated rat liver cells. In: Methods in Cell Biology. 1976. FIG. 23 (page 77). Cell suspension were centrifuged above a 15% buffered metrizamide cushion (density 1.08 gm/cm³) for 60 minutes at 3500 rpm (see FIGS. 17 and 23 in Seglen P O: Preparation of isolated rat liver cells. In: Methods in Cell Biology.).

Kupffer Cell Activation (to Restore Histone Methylation)

Freshly isolated Kupffer cells were activated by overnight incubation in IMDM media containing 10% Fetal bovine serum, antibiotics, 5% WEHI-3B supernatant (containing IL-3) 10 ng/ml murine GM-SCF and 10 ng/ml murine CSF-1.

Antibody Labeling and Fluorescence Microscopy

Antisera used for immunofluorescence and western blotting studies were; anti N- and C-terminus Ikaros [55], anti HP1β/M31 (Serotec) [24], anti lamin B (Santa Cruz), human CREST autoimmune sera, anti-4xmethyl H3-K9, anti mono-methyl H3-K9, anti di-methyl H3-K9, anti tri-methyl H3-K9 and anti methyl H3-K27 [34, 36, 39] anti 1× methyl H3-K9, anti methyl H3-K4, anti acetyl H3-K9, anti acetyl H3-K14 (from Upstate Biotechnologies), anti pan-acetyl H4 (Serotec), anti Enx1 [56], anti EED [57], anti BMI1 [58]. Additional control antibodies used in these analyses were anti HP1β (Euromedex), anti ORC1 (Serotec) and anti PCNA (Sigma).

For IF labeling, cells were attached to glass coverslips pre-coated with poly-L-lysine, washed in PBS and fixed in 2% paraformaldehyde for 10 min. The samples were washed in PBS and quenched with 0.05M NH₄Cl in PBS for 5 min, before further washing in PBS and permeabilisation with 0.3% Triton in PBS for 5 min. Samples were incubated sequentially in blocking solution (0.2% Fish gelatin (Sigma) in PBS) for 30 min and primary antisera (diluted appropriately in blocking buffer with 5% normal goat serum) for 1 hour in a humid chamber. Following washing in blocking solution, samples were incubated for a further 30 min in fluorochrome-labeled secondary antibody (diluted appropriately in blocking buffer and 5% normal goat serum). Slides were washed twice (3 min/wash) in wash buffer, once in PBS alone and mounted in Vectashield (Vector) supplemented with DAPI (0.1 μg/ml). Where goat primary antibodies were utilized, fetal calf serum replaced normal goat serum as a blocking reagent.

Immunofluorescence staining of histone modifications was performed as described [36] with minor modifications. Cells were attached to glass coverslips pre-coated in poly-L-lysine and washed in PBS. Samples were fixed in 2% paraformaldehyde for 10 min, washed in PBS and then incubated in wash buffer for 5 min (wash buffer; PBS, 0.2% BSA, 0.1% Tween20). Preparations were then sequentially incubated, in a humid chamber, in blocking solution (blocking solution; PBS, 10% normal goat serum, 2.5% BSA, 0.1% Tween20) for 30 min, primary rabbit antisera (diluted in blocking solution) for 1 hour and Alexa 488 conjugated goat anti-rabbit IgG(H+L) diluted appropriately in blocking buffer. Samples were mounted in Vectashield supplemented with DAPI and visualized either by confocal microscopy using a TCS-SP1 (Leica Microsystems) or using an Axioplan 2E microscope (Zeiss), Metamorph 4.0 software and images were processed using Adobe photoshop 6.0.

Preparation of Nuclear Extracts from Ex Vivo B Lymphocytes and Western Blot Analyses

For preparing NE-i, NE-s and cytoplasmic (CE) extracts, B cells were washed in ice cold PBS, centrifuged at 600 g for 4 min in a chilled centrifuge (4° C.) and resuspended in ice cold nuclei lysis buffer (10 mM Pipes, pH 6.8, 100 mM NaCl, 300 mM sucrose, 3 mM MgCl2, 1 mM EGTA, supplemented with protease inhibitor cocktail and phosphatase cocktail (Sigma) and 1 mM DTT). Lysis buffer containing 0.75% NP40 was added drop-wise until the concentration of NP40 reached 0.15% and then left on ice for 2 min before centrifugation at 400 g for 2 min. The non-chromatin cytoplasmic fraction (supernatant) was collected and the remaining nuclei were washed once in lysis buffer and centrifuged again as previously. Chromatin was solubilised by DNA digestion with 1 mg/ml of RNAse-free DNAse I (Sigma) in lysis buffer for 30 min at 30° C. NH₄(SO₄) was added from a 1 M stock solution in lysis buffer to a final concentration of 0.25 M. After 5 min on ice, samples were pelleted by centrifuging at 1500 g for 3 min and DNAse I soluble material collected. The pellet of DNAse I insoluble material was then solubilised in Urea buffer (8M urea, 0.1M NaH₂PO₄, 0.01M Tris-HCl pH 8.0) and the protein extracts were quantified and stored at −70° C.

Histone proteins were isolated from whole cells by acid extraction. 1×10⁷ B cells were pelleted and resuspended in 1 ml PBS (4° C.) and centrifuged (500 g for 5 min) and the supernatant removed. Cell pellets were resuspended in 180 μl of ice cold lysis buffer (10 mM HEPES pH 7.9, 1.5 mM MgCl₂, 10 mM KCl, 0.5 mM DTT and 1.5 mM PMSF), 20 μl of 2M HCl added and incubated on ice for 30 min. Following acid lysis the solution was centrifuged 11000 g for 10 min at 4° C., the supernatant of acid soluble proteins collected and sequentially dialyzed against 0.1M acetic acid (twice for 1 hour) and water (1 hour, 3 hours and overnight respectively). The protein solution was quantified and stored at −70° C. Western blotting of protein extracts was carried out as described previously [21].

Results

Global Changes in Chromatin as Lymphocytes Enter the Cell Cycle

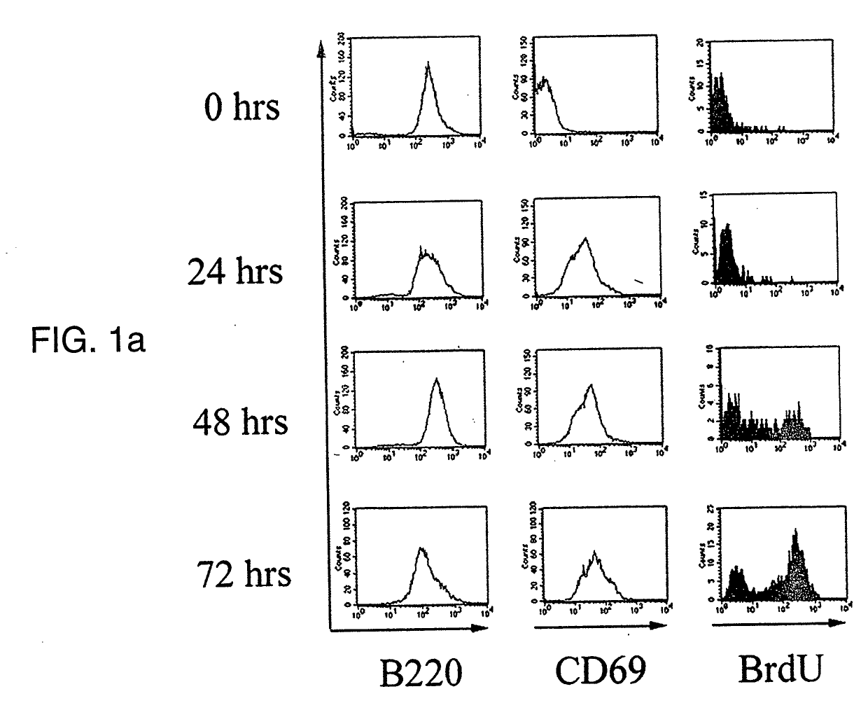

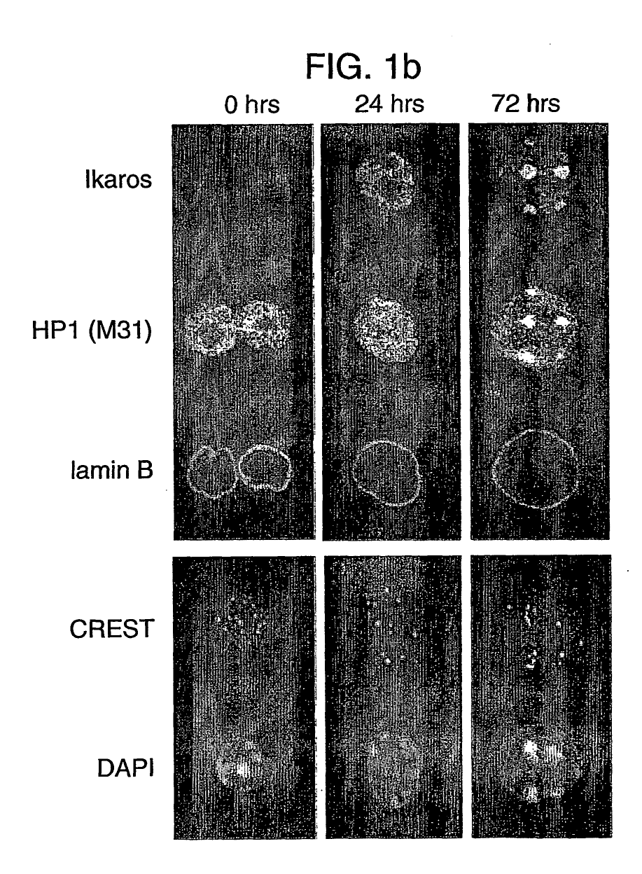

Resting (G₀) B lymphocytes can be purified from the spleen and stimulated to enter the cell cycle and generate progeny in which the correct lineage affiliation and developmental stage is faithfully transmitted. Non-cycling B220⁺ B lymphocytes were isolated from the spleens of normal mice (by CD43 depletion and density gradient separation [22]) and stimulated with anti-IgM, and anti-CD40 in the presence of interleukin-4 (IL-4). Under these conditions cells express the activation marker CD69 within 24 hours and begin DNA synthesis, as detected by BrdU incorporation, 48 to 72 hours after stimulation (FIG. 1a). The distribution of heterochromatin-associated proteins (Ikaros, HP1β and CENP-A) in quiescent and activated cells was monitored by immunofluorescence (IF) and confocal microscopy in which all microscope settings and the laser power were kept constant so that the relative abundance and distribution of proteins could be directly compared (FIG. 1b). In purified resting B cells, Ikaros protein was low or absent but increased following activation and re-located to centromeric domains as reported previously [21]. Low levels of HP1β/M31 were detected in the nuclei of resting B cells. These increased slightly over 24 hours, but HP1β/M31 did not localise to DAPI-intense centromere ‘clusters’ until 48-72 hours after activation. At this time, as lymphocytes began cell division, HP1β/M31 and Ikaros proteins co-localised around centromeric DNA as previously reported [23, 24]. This kinetic re-distribution of Ikaros and HP1β/M31 proteins was confirmed using antibodies specific for alternative regions of these proteins (not shown). This, together with the demonstration that CREST antisera detected centromeres throughout B cell activation (FIG. 1b, lower panels) rules out the possibility that technical problems such as epitope masking or restricted antibody accessibility account for a lack of Ikaros and HP1β detection at constitutive heterochromatin domains in G₀ lymphocytes.

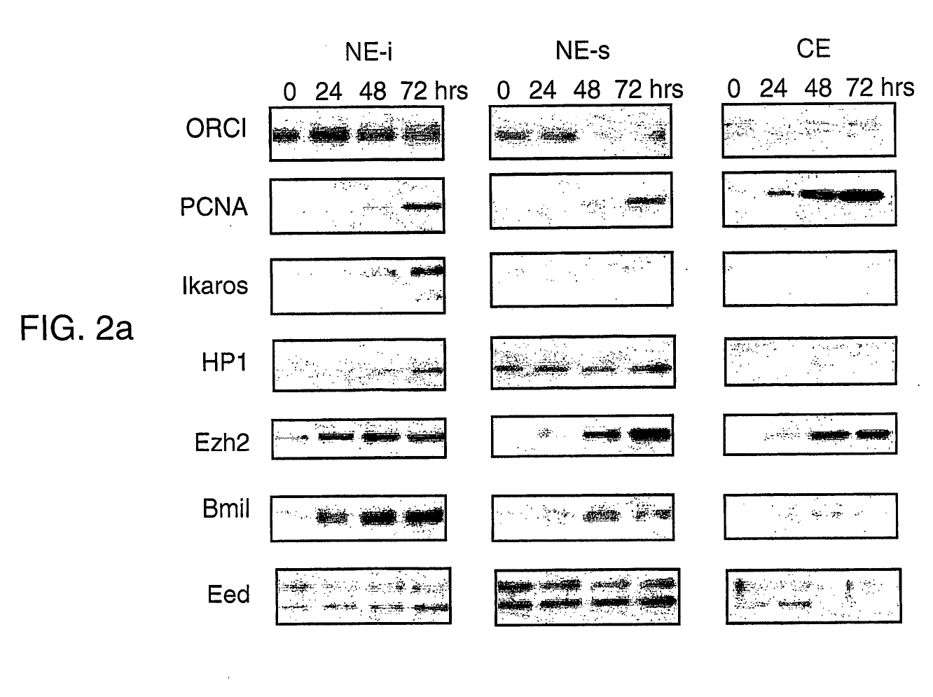

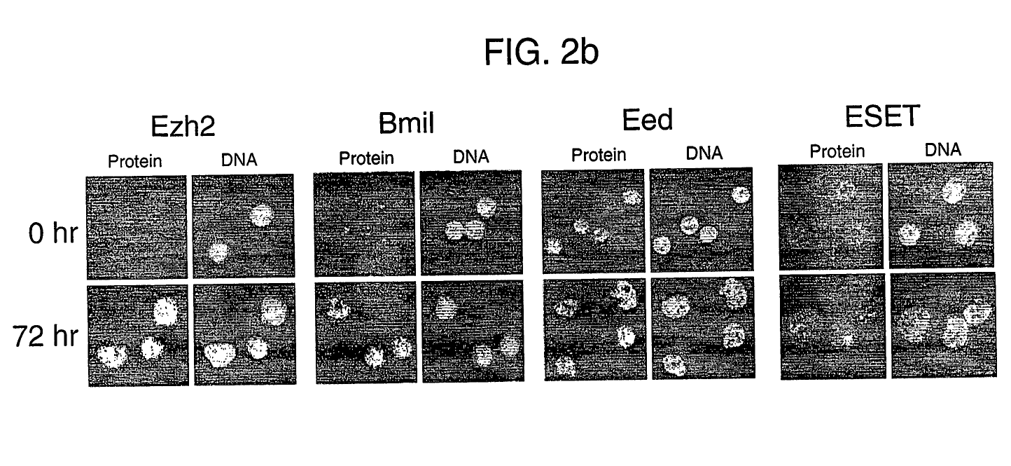

Differences in chromatin composition between resting and activated B cells were also confirmed by western blotting. Nuclei were isolated from resting and activated cells by partial NP40 lysis, a treatment that results in the removal of non-chromatin-bound proteins. Extracts were then subjected to DNase I digestion and soluble (NE-s) or insoluble (NE-i) nuclear fractions were derived and analysed by SDS-PAGE and western blotting (FIG. 2a). Controls included PCNA (a component of the DNA replication machinery synthesized as cells enter S-phase) and ORC1 (a protein which marks origins of replication in quiescent and cycling cells, used here to estimate the equivalence of protein loading). Low levels of PCNA were detected in samples 48 hours after stimulation, becoming more abundant in chromatin fractions after 72 hours. This observation is consistent with most lymphocytes entering S-phase at this time and mirrors the kinetics of BrdU incorporation shown in previous analyses (FIG. 1)[21]. Ikaros proteins corresponding to the major isoforms present in lymphocytes (isoforms I, II, [25]), were absent from G₀ samples (0 hours), but were seen to accumulate in NE-i fractions 48-72 hours after stimulation (FIG. 2a). HP1β/M31 protein was present in the soluble chromatin compartment (NE-s) throughout B cell activation but showed a progressive recruitment to insoluble fractions (NS-i) following activation.

Binding of HP1β to pericentric heterochromatin has previously been shown to depend on the Suv39h histone methyltransferases [26, 27]. The SET domains of Suv39h1 and Suv39h2 catalyse methylation of H3-K9 and provide a high affinity binding site for M31/HP1β [26, 27]. An analogous mechanism probably operates in the recruitment of the Polycomb Repressor Complex-1 (PRC1) to other genomic sites; PRC1 recruitment follows methylation of H3-K9 and H3-K27 by a separate PcG complex that contains the SET domain protein Ezh2, Eed and histone deacetylases [28-31]. In view of these findings we examined the distribution of several additional PcG and HMTase proteins in quiescent and cycling B cells (FIGS. 2a and 2b). Ezh2 and the PRC1-component Bmi1 were selectively upregulated following B cell activation. Both proteins were detected in chromatin-bound and soluble nuclear fractions and their abundance increased following lymphocyte activation (FIG. 2a). In contrast, Eed levels (detected by an antibody that recognizes both putative proteins encoded by two alternatively transcribed mRNAs, [32]) remained relatively unchanged. The selective up-regulation of Ezh2 and Bmi1 PcG proteins was confirmed by IF labeling. Ezh2 staining was low in resting cells but increased in the nucleus of actively proliferating cells (FIG. 2b, 72 hours top panel). Small nuclear foci of Bmi1 were evident in some resting B cells and the intensity and number of nuclear foci increased dramatically upon cell activation. This contrasted with the broadly equivalent nuclear distribution of Eed protein in resting and activated cells. Expression of a ESET, a second SET domain-containing HMTase, also increased following B cell activation and was evident at non-heterochromatic foci (non DNA-dense regions) within the nucleus (FIG. 2b).

H3 Methylation is Reduced in Quiescent B Cells Isolated Ex Vivo

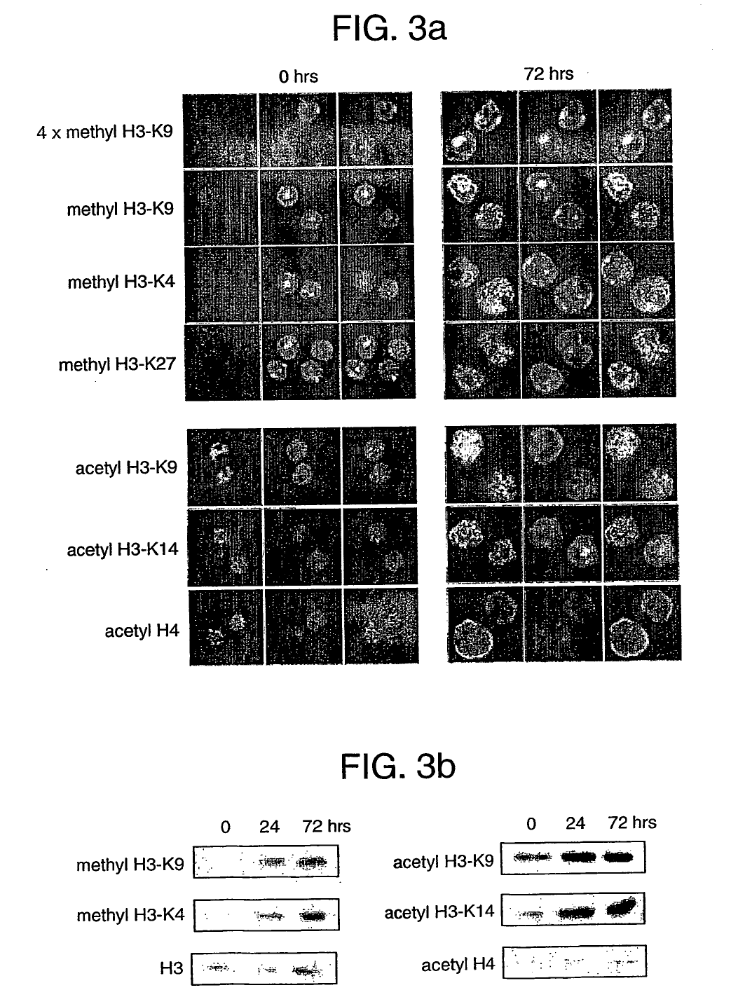

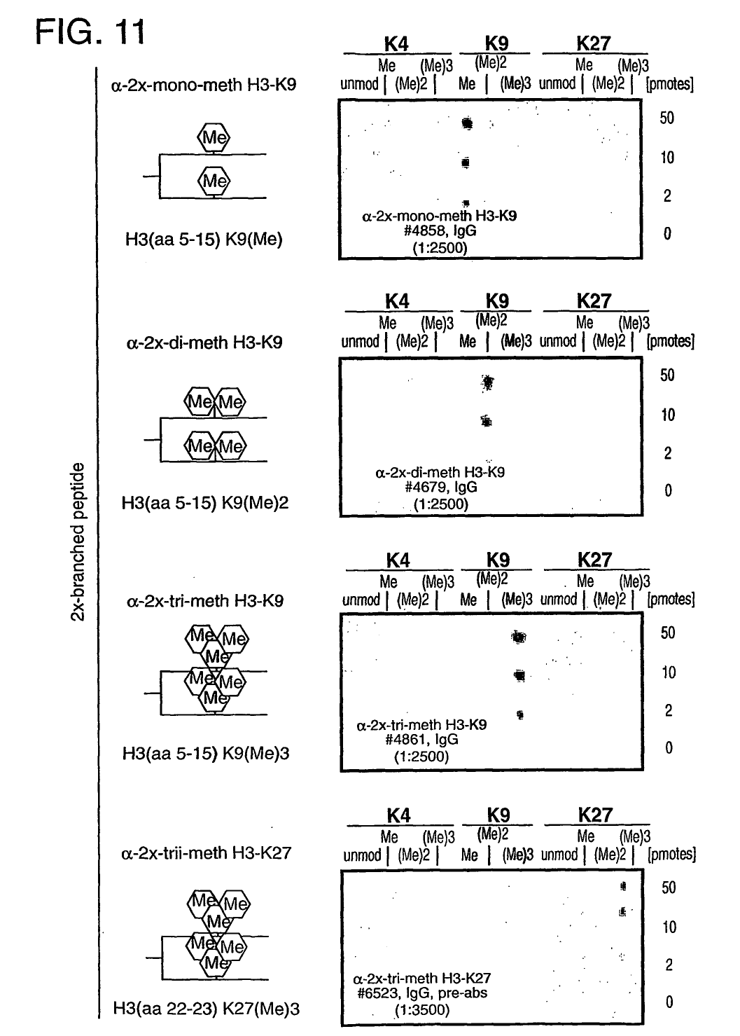

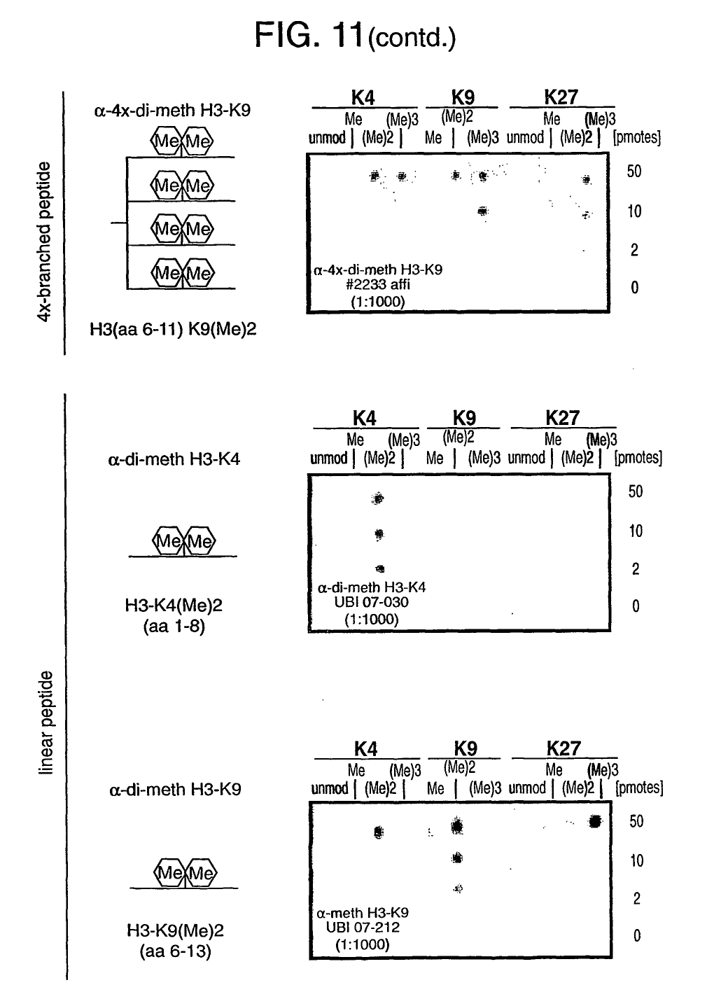

The redistribution of HP1β/M31, Ikaros, Ezh2, Bmi1 and ESET proteins in B cells following mitogenic stimulation parallels the reported nuclear redistribution of genes in response to the activation of quiescent lymphocytes and fibroblasts [21, 33]. One possible explanation for the redistribution of these proteins could be underlying changes in histone methylation. To assess this possibility, the extent of H3-K9 methylation in resting and activated B lymphocytes was assessed by IF and western blotting using anti-methyl H3-K9 antibodies raised against either a branched peptide containing four di-methylated H3-K9 termini (α-4×-di-methyl H3-K9 or 4× methyl), or a single di-methylated H3-K9 terminal peptide (α-di-methyl H3-K9 or methyl H3-K9). The α-4×-di-methyl H3-K9 antibody primarily detects di- and tri-methylated H3-K9 but also has some reactivity against H3-K27 and labels pericentric heterochromatin and some euchromatic sites, whereas the α-di-methyl H3-K9 primarily detects di-methylated H3-K9 (FIG. 11) [34]. Di- and tri-methylated H3-K9 was barely detected in quiescent lymphocytes by IF (FIG. 3a) consistent with very low levels found by western blotting (FIG. 3b). This was surprising since H3-K9 methylation has long been considered a robust modification, which on the basis of the underlying biochemistry has been thought to be almost permanent in nature [1, 35]. Following activation for 72 hours, di- and tri-methylated H3-K9 had become highly abundant within the nucleus of activated lymphocytes (FIG. 3a) and was focussed around DAPI-dense regions, consistent with previous reports [36]. Using a panel of antibodies that recognise alternative lysine residues, methylation of H3-K4, H3-K9, and H3-K27 was low or undetectable in quiescent B cells but substantially increased in cells preparing for division, being routinely detected within 24 hours of activation (FIGS. 3a and b). These data suggest a global reduction in histone methylation in resting B cells in both euchromatic and heterochromatic regions of the genome. In contrast to methylation, acetylation of H3-K9, H3-K14 and H4 appeared broadly similar in resting and activated B cells (FIG. 3a, lower panels and FIG. 3b, right-hand panel). In particular, acetylated H3-K9, H3-K14 and H4 were readily detected in quiescent B lymphocytes. These data show that whereas histone acetylation is robustly retained by quiescent cells, histone methylation appears to be a less stable epigenetic imprint in lymphocytes.

To investigate whether increases in H3-K9 methylation are Suv39h-dependent, we examined B cells from mice lacking both Suv39h1 and Suv39h2 HMTases. B cells from these mice (Suv39h−/−) mice showed a significant increase in euchromatic H3-K9 methylation upon activation, but in contrast to normal cells, no enrichment of heterochromatin-associated H3-K9 methylation (around DAPI intense regions) was evident [36]. Interestingly, Ikaros proteins were focussed at centromeric heterochromatin in activated Suv39h−/− lymphocytes, in the absence of local HP1β/M31 accumulation. This demonstrates that Ikaros binding to pericentric regions is independent of HP1, compatible with recent evidence that whereas HP1 interacts with lysine 9 methylated H3 proteins [26, 27, 37], Ikaros binds directly to repetitive DNA sequences that flank centromeres [38]. These data indicate that the high levels of methylated H3-K9 that typically surround the centromeres of interphase and metaphase chromosomes are not in fact constitutive in B lymphocytes, but are acquired by cells upon entry into cell cycle.

Global Up-Regulation of H3-K9 Methylation in Activated B Lymphocytes

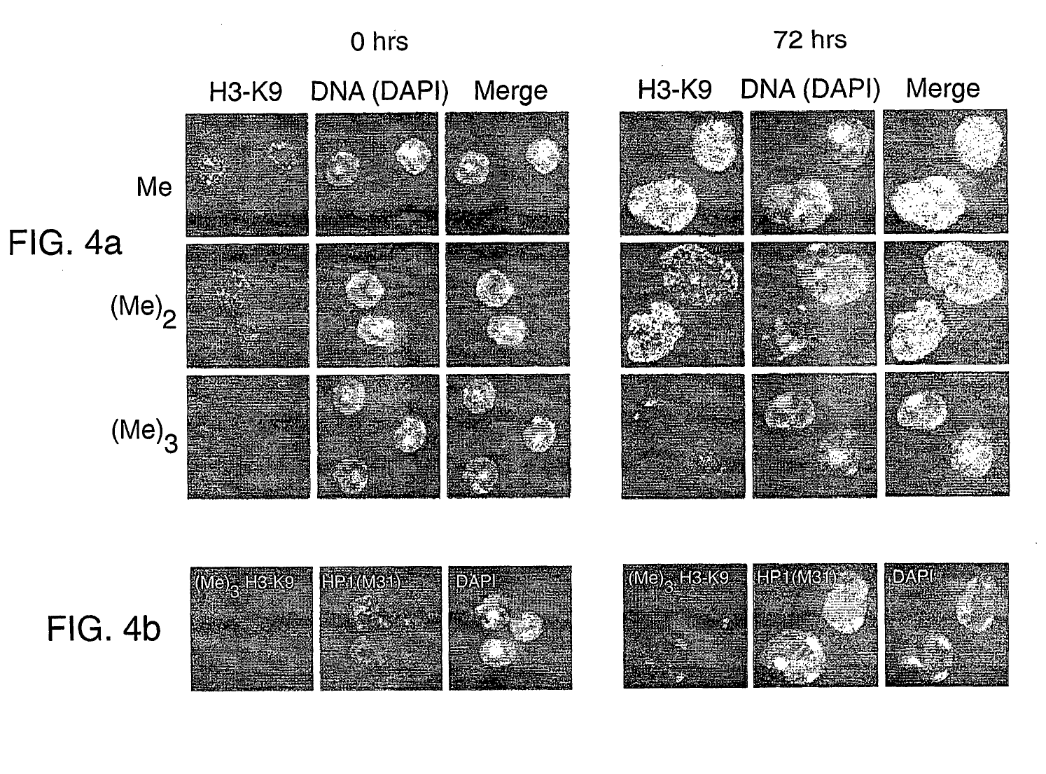

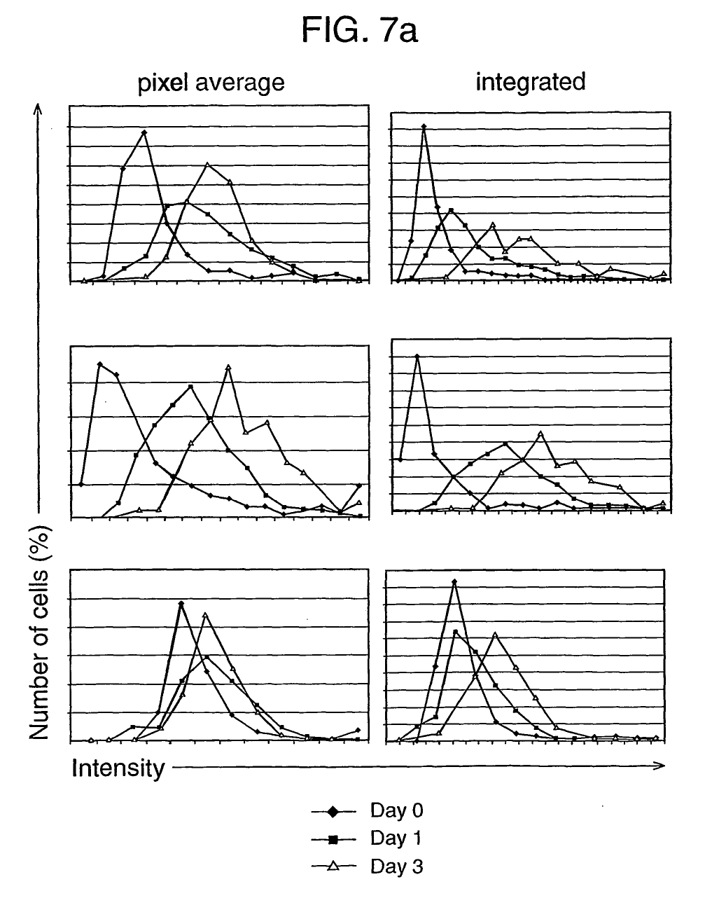

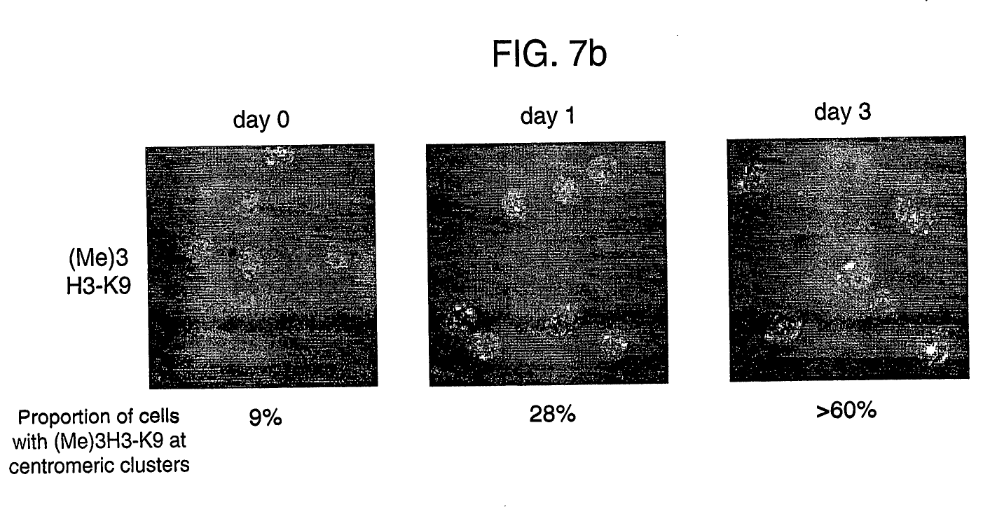

To examine the selectivity of H3-K9 methylation upon B cell activation, antibodies capable of discriminating between the three different states of H3-K9 methylation (mono-, di- or tri-methylated lysine 9) were used to examine quiescent and cycling cells (see FIG. 11). Antibodies used in previous analysis (α-4x-di-methyl H3-K9 and α-di-methyl H3-K9) preferentially recognise di/tri and di methyl H3-K9, respectively, but are inefficient at detecting the mono-methylated state. Using antisera specific for one (Me), two (Me)₂ or three (Me)₃ methyl groups at H3-K9 [39] we consistently observed very low labeling of quiescent B cells, which increased markedly upon activation (FIG. 4a). This was quantified by calculating the average pixel labeling intensity (pixel average) of each nucleus examined or the total intensity of each nucleus (integrated) 0, 1 and 3 days after stimulation (FIG. 7). Tri-methyl H3-K9 labeling was observed only after mitotic stimulation and was confined to discrete locations within the nucleus coincident with DAPI-bright, condensed DNA domains. Confirmation that tri-methyl H3-K9 (FIG. 4b) localised at constitutive heterochromatin in cycling lymphocytes was obtained by co-staining with antibody to HP1β (FIG. 4b). In cycling B cells tri-methyl H3-K9 and HP1β domains routinely co-localised with DAPI-intense regions, an observation that is consistent with reports that HP1β recognises tri-methyl H3-K9 [39].

Hypomethylation of Kupffer Cells in Mouse Liver

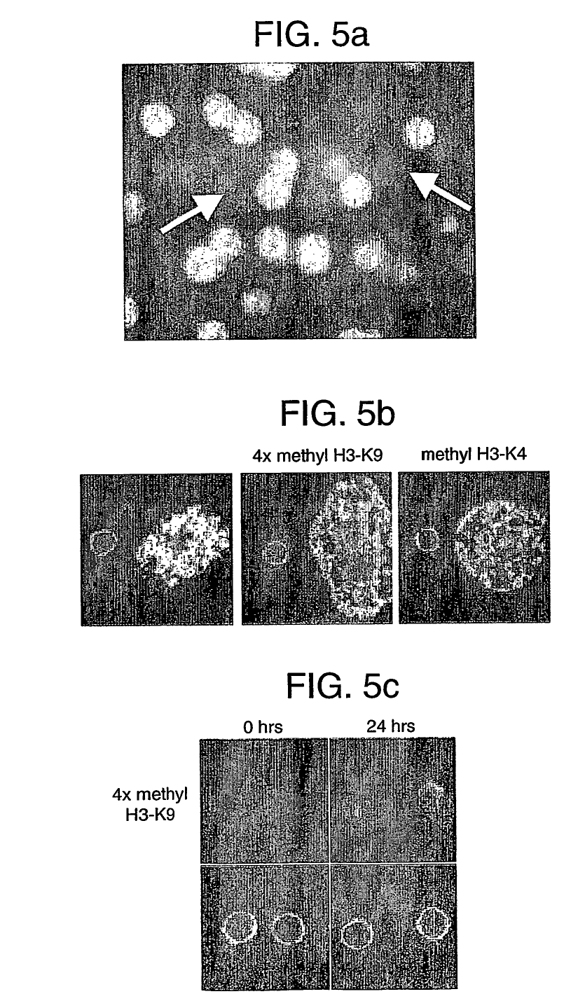

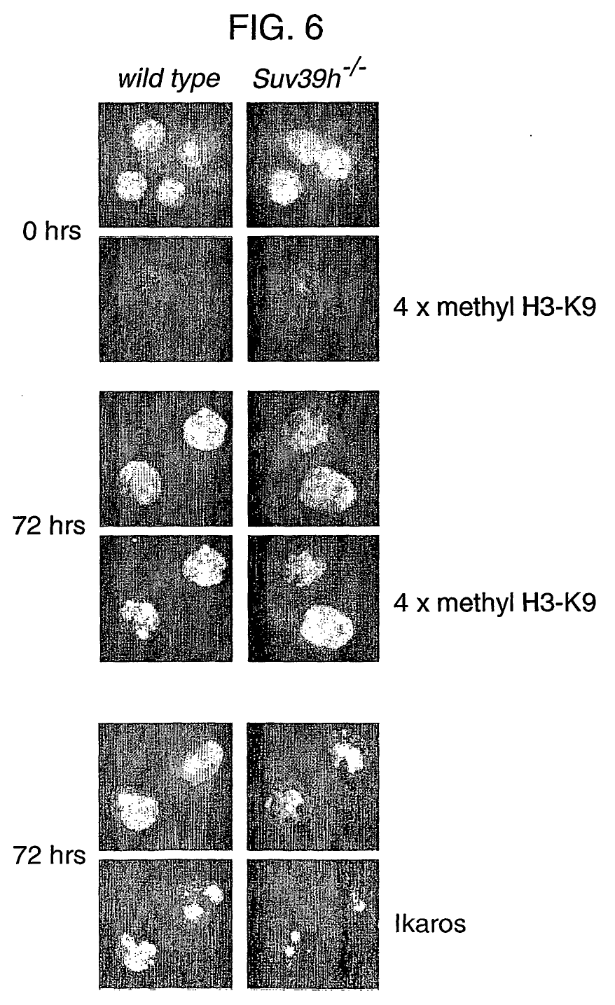

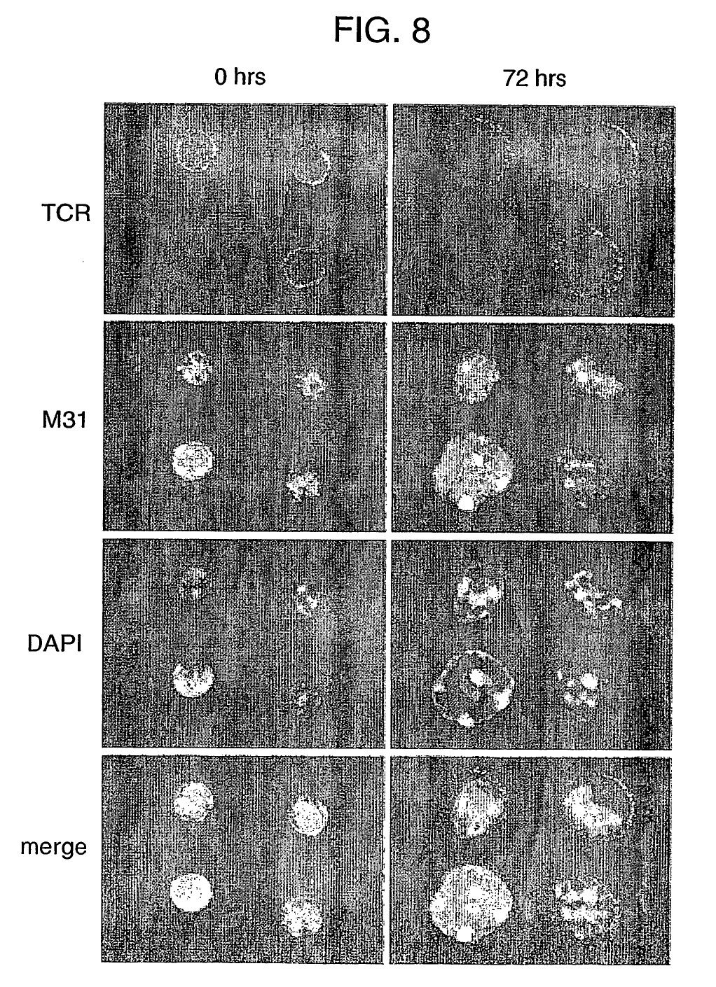

To determine whether reduced histone methylation was typical of other G₀ cell types we also examined ex vivo T lymphocytes and non-cycling cells within the liver. Resting lymph node T cells, identified by T cell receptor (TCR) expression, showed low levels of histone methylation, particularly tri-methylated H3-K9 as recognized by HP1β (see FIG. 8). Liver sections labeled with α-4×-di-methyl H3-K9 (4xmethyl H3-K9) showed evidence of two distinct cell populations. The majority of cells had large nuclei (12-16 μM diameter) and expressed high levels of histone methylation. A second population with smaller nuclei (8-9 μM diameter) lacked H3-K9 methylation (FIG. 5a). The relative abundance of the two cell types was consistent with most cells being hepatocytes and the minority of smaller cells being Kupffer cells. To confirm this we prepared single cell suspensions of murine liver by collagenase treatment using established protocols [40], and identified Kupffer cells on the basis of expression of the leukocyte-specific membrane protein CD45. As shown in FIG. 5b, Kupffer cells expressing surface CD45 (identified by biotinylated anti-CD45 and FITC-avidin) were conveniently discriminated from larger hepatocytes in which endogenous biotin was restricted to the cytoplasm. Co-labeling of liver cell suspensions with 4xmethyl H3-K9 confirmed that the hepatocytes showed high levels of H3-K9 di/tri-methylation while no labeling was apparent in the nuclei of Kupffer cells. Methylated H3-K4 was also detected in hepatocytes but not in Kupffer cells. However, as with resting B cells, H3 hypomethylation was reversed by mitotic stimulation; following overnight culture in the presence of GM-SCF, CSF and interleukin 3 (IL-3), we observed high levels of H3-K9 di/tri-methylation in CD45 positive Kupffer cells (compare upper panels, FIG. 5c). These data provide strong evidence that H3 methylation increases substantially as cells enter the cell cycle.

Discussion and Conclusions

Using a panel of antisera that recognise specific histone methylation states we provide strong evidence of histone hypomethylation in G₀ B lymphocytes. Following mitotic stimulation, histone H3 methylation of lysines 4, 9 and 27 was reinstated in these cells, concurrent with an upregulation and redistribution of several chromatin modifier proteins. Although we cannot exclude the possibility that the lack of observed histone methylation in these G₀ populations is due to an unusual chromatin conformation obstructing the recognition of methylation epitopes, we view this possibility as extremely unlikely for several reasons. Firstly, antisera to different molecular epitopes showed a consistent reduction in histone methylation as judged by IF. Secondly, this observation was confirmed by western blotting—an approach where steric masking of epitopes is not an issue. Thirdly, reduced anti-methyl H3 labeling of resting cells was observed even within presumed euchromatin (recognised by H3-K4) although these regions would be generally considered to be relatively decondensed and accessible. We therefore favor the hypothesis that histone methylation is “lost” or dramatically reduced in quiescent B cells and also in Kupffer cells in mouse liver. An important question is how this loss might be achieved. Several reports have shown that histones are continuously being replaced in cells and that replacement occurs in a replication dependent and independent manner [41, 42]. Cells which remain quiescent for prolonged periods without going through S-phase (for example neurons) have been shown to selectively replace histone H3 with the variant histone H3.3 that, unlike H3, is synthesised throughout the cell cycle [43, 44]. A predominance of the H3.3 variant has been documented in mouse liver [45] and lymphocytes. In lymphocytes, detailed analysis of histone composition during lymphocyte activation suggests that the proportion of H3.3 within the mass pattern of chromatin is directly linked to the length of time in quiescence [46]. More recently, the exchange of H3.1 for H3.3 has been shown at specific loci where replacement appears to be favored or driven by active transcription [47]. Our data would fit with a gradual exchange of H3.1 for H3.3 in quiescent lymphocytes, which together with the lack of expression (or inactivity) of several SET domain proteins (such as Ezh2 and ESET), could result in a global reduction in histone methylation in long-term quiescent lymphocyte populations.

One important consideration is whether the apparent loss of histone methylation that occurs as activated lymphocytes exit the cell cycle, is functionally significant. For example, a consequence of reduced histone methylation in resting cells might be to ‘loosen’ the epigenetic code and effectively enhance cellular plasticity. In principle this could offer an explanation for longstanding claims that some resting (or serum-starved) populations of cells are more efficiently reprogrammed than activated cells [48-50]. To test whether resting B cells are reprogrammed at a higher frequency than activated B cells, we performed nuclear transfer experiments using fertilized embryos as recipients. This allows the potential of different donor nuclei to be assessed in a context where their contribution to embryonic development is not required [51]. As an indicator of plasticity, we compared the extent to which an EGFP transgene [52, 53] that is silent in both resting and active B cells but active from the morula stage onwards (our unpublished data), becomes reactivated after the transfer of lymphocyte nuclei into one-cell embryos.

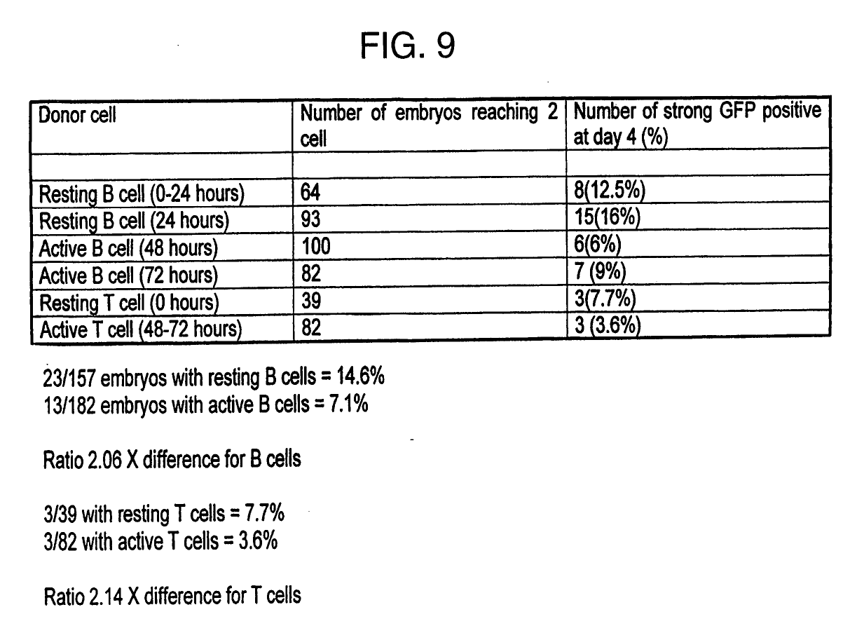

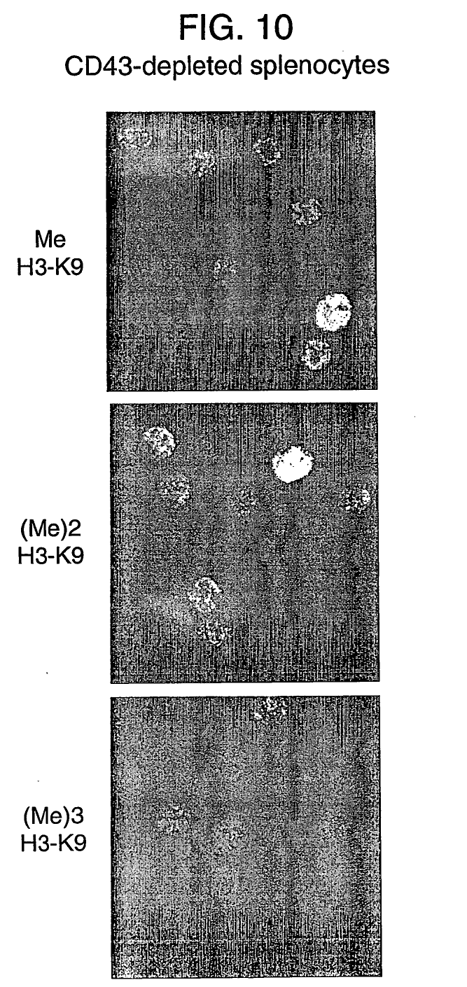

Nuclei from resting (0-24 hours) or active (48-72 hours) B cells carrying the silent EGFP transgene were transferred into fertilised embryos 18-21 hours post injection with human chorionic gonadotrophin (BL6/D2×BL6/D2). Embryos surviving the transfer procedure were cultured overnight in M16 media (Specialty Media). On day one after transfer, 2-cell embryos were transferred into glucose-supplemented CZB media. The majority of operated embryos reached morulae or early blastocyst stage but were somewhat developmentally retarded as compared to non-operated, control embryos. On day four after transfer, GFP expression was assessed. Re-expression of GFP occurred in twice as many fertilized embryos injected with resting B cell nuclei as those injected with 48- or 72-hour activated B cells (FIG. 9). This enhanced performance did not simply reflect a decline in viability of cultured cells since G₀ cells maintained for 24 hours in IL-4 alone (in the absence of mitotic stimulation and upregulation of histone methylation), also showed efficient reversal of transgene silencing (compare 12-16% versus 6-9% GFP-positive embryos, FIG. 9). The data from various experiments is shown in the table in FIG. 9. Similar preliminary results have been obtained with resting and active T cells. These experiments suggest that histone hypomethylation could contribute to the enhanced genomic plasticity of resting cells.

The observation that histone acetylation is robustly retained by quiescent cells whereas histone methylation appears to be a relatively unstable epigenetic trait is intriguing. An explanation for this could be that the basal transcription of active genes in G₀ lymphocytes is sufficient to maintain acetylation of the genome. High levels of histone methylation, in contrast, may only be required only when overall gene activity is increased (for example following mitotic stimulation) to amplify the epigenetic status of a gene prior to DNA synthesis. The finding that histone methylation is a more dynamic epigenetic imprint than was previously anticipated is important. Current views of how a histone code might be interpreted have implied that quality and density of different histone tail modifications in a particular region could be predictive of transcriptional potential. This assumption forms the basis for current Chromatin Immunoprecipitation (ChIP)-based analyses. Here we show that the relative abundance of histone H3 methylation in primary cells differs dramatically between resting and cycling populations. This fact does not negate the concept that histone methylation contributes to cellular memory since relatively low levels of these modifications could still be sufficient to ‘mark’ active or inactive chromatin domains in quiescent cells. However, our demonstration that histone methylation is modulated according to cell cycle status indicates that the density of a particular histone modification cannot simply be equated with transcriptional competence.

### REFERENCES

1. Jenuwein T, Allis C D: Translating the histone code. Science 2001, 293:1074-1080.
2. Berger S L: Histone modifications in transcriptional regulation. Curr Opin Genet Dev 2002, 12:142-148.
3. Jeppesen P, Turner B M: The inactive X chromosome in female mammals is distinguished by a lack of histone H4 acetylation, a cytogenetic marker for gene expression. Cell 1993, 74:281-289.
4. Turner B M: Histone acetylation and an epigenetic code. Bioessays 2000, 22:836-845.
5. Turner B M, Birley A J, Lavender J: Histone H4 isoforms acetylated at specific lysine residues define individual chromosomes and chromatin domains in Drosophila polytene nuclei. Cell 1992, 69:375-384.
6. Rice J C, Allis C D: Histone methylation versus histone acetylation: new insights into epigenetic regulation. Curr Opin Cell Biol 2001, 13:263-273.
7. Strahl B D, Ohba R, Cook R G, Allis C D: Methylation of histone H3 at lysine 4 is highly conserved and correlates with transcriptionally active nuclei in Tetrahymena. Proc Natl Acad Sci USA 1999, 96:14967-14972.
8. Kennison J A: The Polycomb and trithorax group proteins of Drosophila: trans-regulators of homeotic gene function. Annu Rev Genet 1995, 29:289-303.
9. Simon J A, Tamkun J W: Programming off and on states in chromatin: mechanisms of Polycomb and trithorax group complexes. Curr Opin Genet Dev 2002, 12:210-218.
10. Reuter G, Spierer P: Position effect variegation and chromatin proteins. Bioessays 1992, 14:605-612.
11. De Rubertis F, Kadosh D, Henchoz S, Pauli D, Reuter G, Struhl K, Spierer P: The histone deacetylase RPD3 counteracts genomic silencing in Drosophila and yeast. Nature 1996, 384:589-591.
12. Grewal S I, Bonaduce M J, Klar A J: Histone deacetylase homologs regulate epigenetic inheritance of transcriptional silencing and chromosome segregation in fission yeast. Genetics 1998, 150:563-576.
13. Eissenberg J C, Morris G D, Reuter G, Hartnett T: The heterochromatin-associated protein HP-1 is an essential protein in Drosophila with dosage-dependent effects on position-effect variegation. Genetics 1992, 131:345-352.
14. Rea S, Eisenhaber F, O'Carroll D, Strahl B D, Sun Z W, Schmid M, Opravil S, Mechtler K, Ponting C P, Allis C D, Jenuwein T: Regulation of chromatin structure by site-specific histone H3 methyltransferases. Nature 2000, 406:593-599.
15. Grewal S I, Elgin S C: Heterochromatin: new possibilities for the inheritance of structure. Curr Opin Genet Dev 2002, 12:178-187.
16. Aagaard L, Laible G, Selenko P, Schmid M, Dorn R, Schotta G, Kuhfittig S, Wolf A, Lebersorger A, Singh P B, Reuter G, Jenuwein T: Functional mammalian homologues of the Drosophila PEV-modifier Su(var)3-9 encode centromere-associated proteins which complex with the heterochromatin component M31. Embo J 1999, 18:1923-1938.
17. Nakayama J, Rice J C, Strahl B D, Allis C D, Grewal S I: Role of histone H3 lysine 9 methylation in epigenetic control of heterochromatin assembly. Science 2001, 292:110-113.
18. van der Lugt N M, Domen J, Linders K, van Roon M, Robanus-Maandag E, te Riele H, van der Valk M, Deschamps J, Sofroniew M, van Lohuizen M, et al.: Posterior transformation, neurological abnormalities, and severe hematopoietic defects in mice with a targeted deletion of the bmi-1 proto-oncogene. Genes Dev 1994, 8:757-769.
19. Core N, Bel S, Gaunt S J, Aurrand-Lions M, Pearce J, Fisher A, Djabali M: Altered cellular proliferation and mesoderm patterning in Polycomb-M33- deficient mice. Development 1997, 124:721-729.
20. Akasaka T, Tsuji K, Kawahira H, Kanno M, Harigaya K, Hu L, Ebihara Y, Nakahata T, Tetsu O, Taniguchi M, Koseki H: The role of mel-18, a mammalian Polycomb group gene, during IL-7-dependent proliferation of lymphocyte precursors. Immunity 1997, 7:135-146.
21. Brown K E, Baxter J, Graf D, Merkenschlager M, Fisher A G: Dynamic repositioning of genes in the nucleus of lymphocytes preparing for cell division. Mol Cell 1999, 3:207-217.
22. Ratcliffe M J, Julius M H: H-2-restricted T-B cell interactions involved in polyspecific B cell responses mediated by soluble antigen. Eur J Immunol 1982, 12:634-641.
23. Brown K E, Guest S S, Smale S T, Hahm K, Merkenschlager M, Fisher A G: Association of transcriptionally silent genes with Ikaros complexes at centromeric heterochromatin. Cell 1997, 91:845-854.
24. Wreggett K A, Hill F, James P S, Hutchings A, Butcher G W, Singh P B: A mammalian homologue of Drosophila heterochromatin protein 1 (HP1) is a component of constitutive heterochromatin. Cytogenet Cell Genet 1994, 66:99-103.
25. Hahin K, Ernst P, Lo K, Kim G S, Turck C, Smale S T: The lymphoid transcription factor LyF-1 is encoded by specific, alternatively spliced mRNAs derived from the Ikaros gene. Mol Cell Biol 1994, 14:7111-7123.
26. Lachner M, O'Carroll D, Rea S, Mechtler K, Jenuwein T: Methylation of histone H3 lysine 9 creates a binding site for HP1 proteins. Nature 2001, 410:116-120.
27. Bannister A J, Zegerman P, Partridge J F, Miska E A, Thomas J O, Allshire R C, Kouzarides T: Selective recognition of methylated lysine 9 on histone H3 by the HP1 chromo domain. Nature 2001, 410:120-124.
28. Czermin B, Melfi R, McCabe D, Seitz V, Imhof A, Pirrotta V: Drosophila enhancer of Zeste/ESC complexes have a histone H3 methyltransferase activity that marks chromosomal Polycomb sites. Cell 2002, 111:185-196.
29. Muller J, Hart C M, Francis N J, Vargas M L, Sengupta A, Wild B, Miller E L, O'Connor M B, Kingston R E, Simon J A: Histone methyltransferase activity of a Drosophila Polycomb group repressor complex. Cell 2002, 111:197-208.
30. Kuzmichev A, Nishioka K, Erdjument-Bromage H, Tempst P, Reinberg D: Histone methyltransferase activity associated with a human multiprotein complex containing the Enhancer of Zeste protein. Genes Dev 2002, 16:2893-2905.
31. Cao R, Wang L, Wang H, Xia L, Erdjument-Bromage H, Tempst P, Jones R S, Zhang Y: Role of histone H3 lysine 27 methylation in Polycomb-group silencing. Science 2002, 298:1039-1043.
32. Peytavi R, Hong S S, Gay B, d'Angeac A D, Selig L, Benichou S, Benarous R, Boulanger P: HEED, the product of the human homolog of the murine eed gene, binds to the matrix protein of HIV-1. J Biol Chem 1999, 274:1635-1645.
33. Bridger J M, Boyle S, Kill I R, Bickmore W A: Re-modelling of nuclear architecture in quiescent and senescent human fibroblasts. Curr Biol 2000, 10:149-152.
34. Perez-Burgos L, Peters AHFM, Opravil S, Mechtler K, Kauer M, Jenuwein T: Generation and characterisation of anti methyl-lysine histone H3 antibodies. Methods in Enzymology in press.
35. Byvoet P, Shepherd G R, Hardin J M, Noland B J: The distribution and turnover of labeled methyl groups in histone fractions of cultured mammalian cells. Arch Biochem Biophys 1972, 148:558-567.
36. Peters A H, O'Carroll D, Scherthan H, Mechtler K, Sauer S, Schofer C, Weipoltshammer K, Pagani M, Lachner M, Kohlmaier A, Opravil S, Doyle M, Sibilia M, Jenuwein T: Loss of the Suv39h histone methyltransferases impairs mammalian heterochromatin and genome stability. Cell 2001, 107:323-337.
37. Nielsen A L, Oulad-Abdelghani M, Ortiz J A, Remboutsika E, Chambon P, Losson R: Heterochromatin formation in mammalian cells: interaction between histones and HP1 proteins. Mol Cell 2001, 7:729-739.
38. Cobb B S, Morales-Alcelay S, Kleiger G, Brown K E, Fisher A G, Smale S T: Targeting of Ikaros to pericentromeric heterochromatin by direct DNA binding. Genes Dev 2000, 14:2146-2160.
39. Peters AHFM, Kubicek S, Perez-Burgos L, Opravil S, Mechtler K, Kohlmaier A, Beyer A, Tachibana M, Shinkai Y, Jenuwein T: A trilogy of mono-di-tri methylation for histone N-termini at silent chromatin domains. submitted.
40. Seglen P O: Preparation of isolated rat liver cells. In: Methods in Cell Biology. pp. 29-83; 1976: 29-83.
41. Wu R S, Tsai S, Bonner W M: Patterns of histone variant synthesis can distinguish G0 from G1 cells. Cell 1982,31:367-374.
42. Bannister A J, Schneider R, Kouzarides T: Histone methylation: dynamic or static? Cell 2002, 109:801-806.
43. Pina B, Suau P: Changes in histones H2A and H3 variant composition in differentiating and mature rat brain cortical neurons. Dev Biol 1987, 123:51-58.
44. Bosch A, Suau P: Changes in core histone variant composition in differentiating neurons: the roles of differential turnover and synthesis rates. Eur J Cell Biol 1995,68:220-225.
45. Bonner W M, West M H, Stedman J D: Two-dimensional gel analysis of histones in acid extracts of nuclei, cells, and tissues. Eur J Biochem 1980, 109:17-23.
46. Wu R S, Tsai S, Bonner W M: Changes in histone H3 composition and synthesis pattern during lymphocyte activation. Biochemistry 1983, 22:3868-3873.
47. Ahmad K, Henikoff S: The histone variant H3.3 marks active chromatin by replication-independent nucleosome assembly. Mol Cell 2002, 9:1191-1200.
48. Wilmut I, Schnieke A E, McWhir J, Kind A J, Campbell K H: Viable offspring derived from fetal and adult mammalian cells. Nature 1997, 385:810-813.
49. Gurdon J B, Laskey R A, Reeves O R: The developmental capacity of nuclei transplanted from keratinized skin cells of adult frogs. J Embryol Exp Morphol 1975,34:93-112.
50. Kikyo N, Wolffe A P: Reprogramming nuclei: insights from cloning, nuclear transfer and heterokaryons. J Cell Sci 2000, 113 (Pt 1): 11-20.
51. Modlinski J A: Transfer of embryonic nuclei to fertilised mouse eggs and development of tetraploid blastocysts. Nature 1978, 273:466-467.
52. Eggan K, Akutsu H, Hochedlinger K, Rideout W, 3rd, Yanagimachi R, Jaenisch R: X-Chromosome inactivation in cloned mouse embryos. Science 2000, 290:1578-1581.
53. Hadjantonakis A K, Gertsenstein M, Ikawa M, Okabe M, Nagy A: Generating green fluorescent mice by germline transmission of green fluorescent ES cells. Mech Dev 1998, 76:79-90.
54. Huesmann M, Scott B, Kisielow P, von Boehmer H: Kinetics and efficacy of positive selection in the thymus of normal and T cell receptor transgenic mice. Cell 1991, 66:533-540.
55. Hahm K, Cobb B S, McCarty A S, Brown K E, Klug C A, Lee R, Akashi K, Weissman I L, Fisher A G, Smale S T: Helios, a T cell-restricted Ikaros family member that quantitatively associates with Ikaros at centromeric heterochromatin. Genes Dev 1998,12:782-796.
56. Sewalt R G, van der Vlag J, Gunster M J, Hamer K M, den Blaauwen J L, Satijn D P, Hendrix T, van Driel R, Otte A P: Characterization of interactions between the mammalian polycomb-group proteins Enx1/EZH2 and EED suggests the existence of different mammalian polycomb-group protein complexes. Mol Cell Biol 1998, 18:3586-3595.
57. van der Vlag J, Otte A P: Transcriptional repression mediated by the human polycomb-group protein EED involves histone deacetylation. Nat Genet 1999, 23:474478.
58. Gunster M J, Satijn D P, Hamer K M, den Blaauwen J L, de Bruijn D, Alkema M J, van Lohuizen M, van Driel R, Otte A P: Identification and characterization of interactions between the vertebrate polycomb-group protein BMI1 and human homologs of polyhomeotic. Mol Cell Biol 1997, 17:2326-2335.

1. A method of producing an animal embryo, the method comprising transferring from a nuclear donor cell which has been selected on the basis that it is histone hypomethylated at least a portion of the nuclear contents including at least the minimum chromosomal material able to support development into a suitable recipient cell.

2. The method of claim 1 wherein the nuclear donor cell has been selected by experimentally determining that a first cell is histone hypomethylated and selecting a second cell to be which is similar or identical to the first cell to thereby select a histone hypomethylated cell to be used as said nuclear donor cell.

3. The method of claim 2 wherein said first cell and second cell are from the same population of cells.

4. The method of claim 1 wherein the nuclear donor cell has been selected by selecting a cell of a type which has been previously determined as being histone hypomethylated or which has been previously determined as being likely to be histone hypomethylated.

5. The method of claim 2 wherein the level of histone methylation of said first cell or of said cell type when histone hypomethylated is negligible or absent.

6. The method of claim 2 wherein the level of histone methylation of said first cell or of said cell type when histone hypomethylated is assessed on the basis of methylation at one or more residues of H3.

7. The method of claim 2 wherein the level of histone methylation of said first cell or of said cell type when histone hypomethylated is assessed on the basis of methylation at one or more lysine residues.

8. The method according to claim 7 wherein the level of histone methylation is assessed on the basis of methylation at one, two, three or four of the following lysine residues: residues H3, H3, H3K2′ and H3.

9. The method according to claim 8 wherein the level of histone methylation is assessed on the basis of methylation atH3K4 and H3K9.

10. The method according to claim 1 wherein the nuclear donor cell is a mammalian cell.

11. The method according to claim 1 wherein the recipient cell is a mammalian cell.

12. The method according to claim 1 wherein the recipient cell is an enucleated oocyte.

13. A method of producing an animal embryo, the method comprising transferring from a nuclear donor cell at least a portion of the nuclear contents including at least the minimum chromosomal material able to support development into a suitable recipient cell wherein the nuclear donor cell is obtained from an embryo obtained by the method of claim 1.

14. The method according to claim 13 wherein the nuclear donor cell has been selected on the basis that it is histone hypomethylated.

15. A method of producing a foetus, the method comprising allowing an embryo obtained by a method according to claim 1 to develop into a foetus.

16. A method of producing a non-human animal the method comprising allowing an embryo obtained by a method according to claim 1 to develop into said non-human animal.

17. A method of producing an embryonic stem cell line, the method comprising transferring an embryo obtained by the method of claim 1 to a culture system.

18. A method of producing an embryonic stem cell line, the method comprising isolating the inner cell mass of an embryo obtained by the method of claim 1 and transferring the inner cell mass to a culture system.

19. A method according to claim 1 wherein the nuclear donor cell is a non-human cell.

20. A method according to claim 1 wherein the recipient cell is a non-human cell.

21-33. (canceled)
